(19) United States
(12) Patent Application Publication
(10) Pub. No.: US 2024/0390581 A1
(43) Pub. Date: Nov. 28, 2024

(54) METHOD AND APPARATUS FOR A STEADY STATE INTRAMUSCULAR INFUSION OF SEROTONERGIC DRUGS

(71) Applicant: PSOULMED, LLC, Plymouth, MN (US)

(72) Inventors: John M. BILLINGS, Plymouth, MN (US); Farhad JAFARI, Minneapolis, MN (US)

(73) Assignee: PSOULMED, LLC, Plymouth, MN (US)

(21) Appl. No.: 18/576,658

(22) PCT Filed: Jul. 6, 2022
(86) PCT No.: PCT/US2022/036195

§ 371 Date: Jan. 4, 2024

Related U.S. Application Data
(60) Provisional application No. 63/292,209, filed on Dec. 21, 2021.
(60) Provisional application No. 63/219,170, filed on Jul. 7, 2021.

(51) Int. Cl. A61M 5/168; A61M 5/158; A61M 5/172

(52) U.S. Cl. CPC A61M 5/16877; A61M 5/158; A61M 5/172

(57) ABSTRACT

Methods and apparatus for introducing a steady state concentration of a psychotropic drug are provided. The apparatus comprises a computer assisted diffusion pump that allows for the maintenance of a desired concentration of the drug for an extended period of time. The apparatus also provides a modified needle for intramuscular administration of the drug. The method allows for treatment of a variety of indications including stress, depression, anxiety and insomnia as well as addictive disorders including alcohol, cigarette addiction and drug addiction.

### CROSS-REFERENCE TO RELATED APPLICATIONS

This application claims benefit of priority from U.S. Provisional Application Ser. No. 63/219,170 filed on Jul. 7, 2021, and U.S. Provisional Application Ser. No. 63/292,209 filed on Dec. 21, 2021, each or which is incorporated herein by reference in their entirety.

### FIELD OF THE INVENTION

The present invention provides methods and apparatus for the steady state infusion of psychedelic drugs such as N,N-dimethyltryptamine (DMT).

### BACKGROUND OF THE INVENTION

N,N-dimethyltryptamine (DMT) is a psychedelic molecule that is produced in plants and mammals, that is believed, but not proven to be synthesized in the human brain endogenously. When given in sufficient doses exogenously, DMT produces profound psychedelic states that are often described as indescribable and transcendent. Such effects are also identified with the use of psychedelics such as psilocybin, lysergic acid diethylamide (LSD) and mescaline, for example.

Recent studies have indicated that in addition to altered states, DMT experiences can also help to ameliorate a host of mental health issues including stress, depression, anxiety and insomnia as well as addictive disorders including alcohol and cigarette addiction.

The half-life of DMT is not precisely known and likely depends on its mode of ingestion. For example, the half-life of injected DMT is thought to be about 15 minutes while oral ingestion has virtually no effect due to its rapid metabolism by monoamine oxidase. Another confounding factor with DMT injection is that as a serotonergic, DMT is vasoconstrictive and may result in intravenous thrombophlebitis.

Achieving psychedelic dissociation is of interest to the scientific community, as it would help to characterize the 5-HT2a (serotonin) receptor system, help us understand what it does, and possibly, why it exists. Furthermore, 5-HT2a receptor agonists, particularly drugs described as having psychedelic or entheogenic properties, may have therapeutic use, particularly with the treatment of disorders ranging from major depression, post-traumatic stress, existential distress, addiction and potentially more. Recently, the FDA announced psilocybin, another 5-HT2a agonist, as a “breakthrough therapy” for depression. Achieving an extended state or steady state DMT experience, may allow for DMT to have more therapeutic utility, and may offer the convenience of allowing that administration to fit conveniently within a therapeutic clinical schedule, where the state can be achieved within minutes, and the experience can be stopped at any time, with the drug effects fulling wearing off in approximately an hour.

Thus, there is a need for new methods and apparatus, which can be used to provide steady state levels of serotonergic psychotropes for treatment and medical investigation.

### SUMMARY OF THE INVENTION

Provided herein are methods and apparatus which allow infusion of steady state levels of drugs for treatment and investigation.

Therefore, in one embodiment according to the invention, provided is a method for steady state infusion of a drug comprising: determining the rate constants for transport of a drug from an infusion site to an active site; creating a computer deliverable program that calculates individual rate constants used for subsequent passes. These rate constants are then programmed to control an infusion pump; providing an infusion pump controllable by the computer deliverable program; and delivering the drug to a patient via the computer directed infusion pump at a desired flow rate or flow constant (k). In various exemplary embodiments, the delivery is intravenous or intramuscular. In some embodiments the drug is a serotonergic drug. In these and other embodiments, the serotonergic drug is a psychedelic drug or a psychotropic drug. In some embodiments, intramuscular infusion comprises a needle having beveled tip, with a hole at the end, typical of needles used for intramuscular injection. In yet other aspects, the needle comprises a number of side holes ranging from 1-100. In various embodiments, the needle comprises 2-20 side holes. In some embodiments, the needle comprises curve at the base of the needle ranging from 0 to 90 degrees. In still various embodiments an initial bolus of the drug is delivered followed by the steady state delivery. In these embodiments, the computer deliverable program comprises a two-compartment model or a multicompetent model. In aspects, the pump is a miniature pump. In some exemplary embodiments, the pump is an implantable pump. In various embodiments, intramuscular means within the pectoralis, the gluteus, the calf, the rectus abdominus, the quadriceps or other muscle capable of maintaining an intramuscular needle.

In yet other exemplary embodiments, disclosed herein is a method of treating a mental health indication comprising administering to a patient in need thereof: a steady state concentration of a psychotropic compound, wherein the steady state concentration is maintained by a by a computer controlled diffusion pump wherein the diffusion pump maintains the steady state concentration according to two compartment or multi-compartment kinetics. In some embodiments, the kinetics is first order. In various other embodiments, the kinetics is second order. In these and various other embodiments, the psychotropic compound is administered intravenously. In yet other embodiments, the psychotropic compound is administered intramuscularly. In various embodiments, modified needle is used. In some embodiments, the modified needle is an indwelling cannula. In some aspects the needle comprises 2-200 side holes. In various aspects the needle comprises 2-20 side holes. In various embodiments, the mental health indication is: stress, depression, anxiety, insomnia, obsessive-compulsive disorder and addictive disorders. In yet other embodiments, the addictive disorders are: substance abuse. In aspects, the substance abuse is abuse of drugs, cigarettes, media.

In still other exemplary embodiments, disclosed herein is an apparatus for administering a steady-state infusion of a drug comprising: a computer assisted infusion device; a computer program capable of driving the infusion device; wherein the computer program calculates the rate constants of a patient using the infusion device; and wherein the infusion device delivers a steady-state infusion of drug to the patient. In various exemplary embodiments, the infusion device delivers the drug intravenously or intramuscularly. In some embodiments, intramuscular infusion comprises a modified needle. In various embodiments the modified needle comprises a beveled tip with a hole at the end. In aspects, the needle comprises a number of side holes ranging from 2-200 to 2-100 to 2-20. In various aspects, the needle comprises curve at the base of the needle ranging from 0 to 90 degrees. In these embodiments the computer assisted infusion device infuses the drug with kinetics of a two-compartment model or a multi-compartment model.

In still other exemplary embodiments, disclosed herewith is a method for decreasing intravenous thrombophlebitis resulting from infusion of serotonergic compounds. In various embodiments, the infusion is intravenous. In yet other embodiments, the infusion is intramuscular. In various exemplary embodiments, intramuscular means within the pectoralis, the gluteus, the calf, the rectus abdominus, the deltoid or the quadriceps. Of course, those of skill in the art will appreciate that any muscle capable of maintaining an intramuscular needle is acceptable for use in the method.

In yet other exemplary embodiments, disclosed is a method for adding low concentration heparin in doses ranging from 0.1-100 IU per mL. In various embodiments, the heparin concentration is in the range of 1-10 IU per mL.

In yet other exemplary embodiments, provided is a method for decreasing venous injury and improving flow rate in a patient infused with a serotonergic compound comprising adding magnesium in doses ranging from 0.1 mg/mL to 100 mg/mL, to the infusate. In one aspect the magnesium is added in the range of about 1-2 mg/mL, to the DMT infusion directly. In some embodiments, the magnesium is added to a maintenance line.

In yet other exemplary embodiments, provided is a method for improving vein patency and dilution from blood flow as well as normal saline comprising preparing the vein to be cannulated with nitroglycerine ointment prior to gaining intravenous access. In some aspects, heparin is added to the infusate in doses ranging from 0.1-100 IU. In some aspects, the heparin is added in doses of 1-10 IU. In other aspects, the heparin is added to the maintenance line. In still other embodiments magnesium is added to the infusate in doses ranging from 0.1-100 IU. In various aspects, the magnesium is added in doses of 1-10 IU. In various aspects the magnesium is added to the maintenance line.

In yet other exemplary embodiments provided herewith is a method for creating an injectable form of a serotonergic compound salt such as fumarate, or freebase of a serotonergic compound in a solution or suspension or emulsion comprising: an iso osmolar or near iso osmolar carrier, minimizing molecular interaction with the walls and endothelium of the peripheral vasculature, while maintaining water soluble or miscible properties as to not adversely affect the pharmacokinetics. In various embodiments, the iso-osmolar or near iso-osmolar carrier comprises hyaluronic acid or polyethylene glycol.

In still other exemplary embodiments, disclosed herein is a method for creating an injectable form of any other drug or substance that may cause venous irritation, to minimize the risk of thrombophlebitis, such as nanoparticles or crystals in suspension, or similarly, chemotherapeutic drugs, anesthetic agents and the like.

While multiple embodiments are disclosed, still other embodiments of the present invention will become apparent to those skilled in the art from the following detailed description. As will be apparent, the invention is capable of modifications in various obvious aspects, all without departing from the spirit and scope of the present invention. Accordingly, the detailed descriptions are to be regarded as illustrative in nature and not restrictive.

### BRIEF DESCRIPTION OF THE FIGURES

Various exemplary embodiments of the compositions and methods according to the invention will be described in detail, with reference to the following Figures wherein:

FIG. 1, is a prior art diagram of a two-compartment transport model according to Gallimore-Strassman (G-S);

FIG. 2, is a diagram of a multi-compartment transport model according to one embodiment of the invention;

FIG. 3, Constant forcing function: Evolution of concentrations in the G-S Model: Constant Infusion Rate. [C]=cerebrospinal fluid, [M]=muscle, [P]=periphery.

FIG. 4, On/Off/On forcing function: Evolution of concentrations in the Prior Art (G-S) model: 1 min on, 3 min off. [C]=cerebrospinal fluid, [M]=muscle, [P]=periphery.

FIG. 5, Concentration Evolution for Linear Case with Random kᵢ Initialization. Relative concentrations based on Example 1 rate constants. [M]=muscle, [B]=blood, [P]=periphery, [C]=cerebrospinal fluid.

FIG. 6, Linear Case, F (t)=(1, 0, 0, 0), Evolution of concentrations;

FIGS. 7A-71, Extraction of the rate constants, the values of the rate constants extracted from the concentrations in the 4 compartments, as displayed in FIGS. 5 and 6; 7A=k₀, 7B=k₁, C=k₂, D=k₃, E=k₄, F=k₅, G=k₆, H=k₇, I=k₈,

FIG. 8A, is a diagram of a computer controlled intramuscular pump connected to a patient according to one embodiment of the invention. 10 computer, 12 computer connection to blood pressure compressor, 14 syringe, 16 syringe pump, computer connection to oximeter, 22 compressor connection to blood pressure cuff, 24 microbore tubing connecting cannula to syringe pump, 26 electroencephalogram leads, 28 blood pressure cuff. 8B is a close up of the pump/syringe connection shown in FIG. 8A.

FIG. 9, is a schematic of data acquisition according to one embodiment of the invention.

### DETAILED DESCRIPTION OF THE EXEMPLARY EMBODIMENTS

Tryptamine is an indolamine metabolite of the essential amino acid, tryptophan. The chemical structure is defined by an indole—a fused benzene and pyrrole ring, and a 2-aminoethyl group at the third carbon. Serotonin and other aminergic neuromodulators or psychedelics including DMT, psilocybin, LSD and psilocin among others are synthesized from tryptophan.

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

In the specification and in the claims, the terms “including” and “comprising” are open-ended terms and should be interpreted to mean “including, but not limited to . . . .” These terms encompass the more restrictive terms “consisting essentially of” and “consisting of.”

It must be noted that as used herein and in the appended claims, the singular forms “a”, “an”, and “the” include plural reference unless the context clearly dictates otherwise. As well, the terms “a” (or “an”), “one or more” and “at least one” can be used interchangeably herein. It is also to be noted that the terms “comprising”, “including”, “characterized by”, “contain(s)” and “having” and variants thereof can be used interchangeably and are intended to be open-ended transitional phrases, terms, or words that do not preclude the possibility of additional acts or structures.

“Biological activity” and its contextual equivalents “activity” and “bioactivity” means that a compound elicits a statistically valid effect in any one biological test assays. Preferably, the threshold for defining an “active” compound will be reproducible and statistically valid effects of at least 25% deviation from untreated control at concentrations at or lower than 1 μM.

“Transport” as used herein refers to both active transport and passive transport, i.e. diffusion. Passive transport (diffusion) is the flow of molecules down a concentration gradient. The movement of molecules from an area of high concentration to low concentration.

“Subject” means living organisms susceptible to conditions or diseases caused or contributed to by infection, inflammation, inflammatory responses, vasoconstriction and myeloid suppression. Examples of subjects include humans, dogs, cats, cows, goats and mice. The term subject is further intended to include transgenic species such as, for example, transgenic mice.

As used herein, infusion pump refers to a pump that delivers drugs, hormones and other test compounds at continuous and controlled rates that may extend from days to months. In various embodiments, the pump may be small and implantable. In other embodiments, the pump may be an external pump. In various embodiments, the pump may be computer controlled and/or include a feedback loop. In some instances, such pumps may be referred to as osmotic pumps. Infusion pumps are commercially available from, for example, Baxter, Medfusion and Alaris to name a few.

As used herein the term “flow rate” refers to the mass of a substance, which passes per unit of time. As used herein, the term “rate constant” is the proportionality constant in the equation that expresses the relationship between the rate of a chemical reaction and the concentrations of the reacting substances.

As used herein, the term “steady-state” delivery refers to a steady-state concentration (Css) which occurs when the amount of a drug being absorbed is the same amount that being cleared from the body when the drug is given continuously or repeatedly. Css is the term when the concentration of the drug in the body stays constant.

“Patient” and “Subject” are used interchangeably herein to refer to an individual who is treated or administered with a drug or composition as described herein.

Another way to approximate the concentration of drug at an effect site is through subject reported subjective experience. In the example of pain medication, this could be a pain rating. In the case of DMT or other serotonergic compounds, this can be described as sub threshold, threshold, ideal, and above ideal, which can be fed into the model as an estimate for the effect site concentration. This estimate is useful because the rate constants that are derived will allow a similar concentration (whatever it may be), and thus similar effect to be achieved in a steady state or near steady state.

“Psychotropic” as used herein means any drug that affects behavior, mood, thoughts or perception. The term “psychedelic” refers to drugs that induce new or altered perceptions or sensory experiences. Generally, psychotropic and psychedelic are used interchangeably. Such drugs are thought to act through the serotonin (5HT) 2A receptor system. Such “tryptamines” include but are not limited to psilocin, psilocybin, bufotenin, baeocystin, aeruginascin, 5MeO-DMT, N,N-Dimethyltryptamine (DMT), 5-Bromo-DMT, to name a few.

As used herein the term “aminergic” refers to neurons and neural pathways that utilize amines (especially monoamines other than epinephrine) as neurotransmitters.

“Tryptamine” as used herein refers to compounds that orientate from the decarboxylation of tryptophan from which the neurotransmitter serotonin is synthesized.

Micro-dose refers to the ingestion of near-threshold perceptible doses of psychedelic substances.

As used herein, the term “intravenous” refers to a drug being administered into a vein. The term intramuscular refers to a drug being administered into a muscle.

As used herein, when referring to a drug, “administer” or administering” means introducing a drug into the body via buccal, suppository, intravenous, intramuscular or oral or other means.

DMT has a very short biological half-life, in the range of minutes, and experiences from inhaling vaporized or smoked DMT, as well as a single IV bolus, typically results in an experience that lasts around 10-15 minutes or less, with the peak experience typically around 5 minutes. The user experience is often described as being transported to another world or space; however, this can be overwhelming and difficult to navigate, in part due to the very short half-life. This short half-life also presents a challenge to researchers who wish to monitor brain activity during a DMT experience.

Those of skill in the art will appreciate that any analysis of the kinetics of flow of an active compound from its site of introduction to its site of action depends on the barriers to transport of the compound from introduction to action. In the present case, it is well appreciated that the site of action of psychotropic compounds is the brain. Any exact site of action in the brain is still not presently understood in full and is therefore a good question for any research. However, assuming the substance freely passes the blood brain barrier (BBB) and that it is not metabolized into further active compounds (as opposed to being metabolically deactivated) the kinetics of the active compounds depends on its transport from the site of administration to its site of action.

Because psychotropic compounds pass freely through the blood brain barrier, intravenous administration of such compounds can be modeled on by a two-compartment model. That is simply the flow of the drug from the blood to the brain. However, as disused above, because serotonergic drugs like the tryptamines may be thrombophlebitic their administration intravenously may be detrimental in single dose cases and dangerous in cases where an a steady state is desired or indwelling needle or cannula is used. Thus, an intramuscular administration may be desirable. In cases of intramuscular administration of serotonergics, a two-compartment model is incapable of representing the kinetics of transport from the site of administration to the site of action. Thus, in the case of intramuscular administration, a multicompetent model is required.

It will be appreciated that intramuscular administration requires modeling of the active compound from a site within a muscle, through a cell membrane, through a capillary membrane and into the blood. It is unclear if it is necessary from such drugs to then pass through serum into the blood cell and such transport will happen in any case as compounds diffuse down a concentration gradient. However, such compounds will continue to diffuse down a concentration gradient from the site of administration throughout the path of the blood flow until reaching an equilibrium (in the case of continuous steady state infusion) or until the drug is eliminated. In any case, transport of the drug from the blood to the brain then still requires the transport back through a capillary membrane in the brain, potentially into the CSF and transport through brain cells or into neurons. Thus, modeling of a multi-compartment system is required in order to understand more perfectly the actions of the psychotropic compounds. Moreover, it will be appreciated that steady-state infusion requires an understanding of the kinetics of transport across each compartment.

### Background and Previous Work

The disclosure provided herein is intended to be quite general and to apply to many drugs and target delivery systems. This work was initiated to model the delivery of DMT subsequent to intravenous or intramuscular application of this drug in solute form. Although the intramuscular administration of DMT has not previously been used, the analysis of previous methods set forth below identifies why an intramuscular delivery of DMT is useful in therapeutic settings.

One of the first attempts to collect human data on the effects of hallucinogenic drugs was made in 1994 “Dose-Response Study of N,N-Dimethyltryptamine in Humans” (Strassman and Qualis, Arch Gen Psychiatry. 1994; 51(2):98-108. doi:10.1001/archpsyc.1994.). In this study, investigators studied the effect of a single bolus of graded doses. They found psychological effects of IV DMT began almost immediately after administration, peaked at 90 to 120 seconds, and were almost completely resolved by 30 minutes.

Other investigators posited the question; is there a delineation between an experience that originates from the brain, like a dream, or from an external stimulus, akin to the visual or auditory cortex being stimulated by neural receptors in the eyes and ears? To this end, a study was conducted to investigate a steady-state DMT experience via continuous IV infusion modeled after the data collected in the 1994 study. (Strassman, R. J. (1994). Dose-response study of N,N-dimethyltryptamine in humans. Archives of General Psychiatry, 51(2), 85.) The authors proposed that the kinetics can be approximated to a 2-compartment model with Michaelis Menten Kinetics, which requires continuous dose adjustment for 20 minutes, followed by a steady state, where the dose no longer requires adjustment. These kinetics are made more obtainable because DMT (and most serotonergic drugs) pass freely through the blood brain barrier and there are no active metabolites.

Another unforeseen problem with the proposed model of a steady state infusion is that DMT is serotonergic and therefore vasoconstrictive at and near the site of peripheral IV insertion. This in combination with probable chemical irritation, even with iso-osmolar concentrations buffered to a physiologic pH (using DMT fumarate or similar salt), intravenous thrombophlebitis can occur at a rate which would be unacceptable to conducting human trials.

In 2016, Gallimore and Strassman published a paper in Frontiers of Pharmacology entitled: A model for the application of target-controlled intravenous infusion for a prolonged immersive DMT psychedelic experience (Article 211, Volume 7, July 2016). Unfortunately, this paper is full of mathematical errors which are examined in Example 1 herein.

### Intravenous Model

A second, and perhaps more serious issue, with the Gallimore Strassman paper is the administration of DMT intravenously. Based on empirical and investigative data it appears that the serotonin receptors in the venous walls interact with DMT, causing vasoconstriction, phlebitis and eventual closing of the infusion veins, and possibly resulting in permanent venous injury. This reaction introduces a significant concentration dependence of the rate constants at the infusion site, affecting the transport of the drug to the blood stream and makes the model significantly more complicated, if not completely inapplicable or clinically hazardous. This is especially true of serotonergic drugs which, as noted, can be phlebitic at the site of administration. This in combination with probable chemical irritation, even with iso-osmolar concentrations buffered to a physiologic pH (using DMT fumarate or similar salt), intravenous thrombophlebitis can occur at a rate

Those of skill in the art will appreciate that what is lacking is a method for achieving steady state of an infused drug such as DMT via continuous or intermittent intravenous or intramuscular infusion, via a model based on known parameters of that drug, and feedback of patient experience into this model for additional fine-tuning. Thus, one goal met herein provides:

1. The corrected method and kinetics to achieve steady state IV infusion in humans;

2. Methods for decreasing intravenous thrombophlebitis related to DMT infusion, or any substance that predisposes to thrombophlebitis;

3. A method for achieving steady state infusion of DMT via continuous intramuscular injection; and

4. A method for performing infusions of other drugs or compounds via continuous intramuscular infusion, which avoids venous cannulation, and thus risk for vascular damage via thrombophlebitis.

In the following, first order kinetics are used to model the concentration of a drug injected into a system, such as the human body, via either intravenous bolus and/or infusion, or via intramuscular bolus and/or infusion, as it transitions into the bloodstream, and from there, to a target organ or effect site. During its transport the drug is metabolized and/or eliminated at different rates from the muscle and the blood stream leading to reduction of the amount reaching the target organ or effect site. The primary goal is to derive the concentration of the drug in the target organ or effect site, in the presence of different infusion regimens, and to derive optimal approaches to arrive at steady-state or near steady-state concentrations in the target organ or effect site, for different ranges of parameters (rate constants).

More significantly, the model allows the determination of the rate constants (both constant and time dependent rate constants depending on the concentrations) for the drug transport between the different compartments. There are a number of ways of determining the evolution of concentration in each compartment. Serial venous blood draws is a common example, and other methods include, but are not limited to invasive procedures such as cerebrospinal fluid collection, or other organ or fluid collection, and noninvasive procedures such as advanced imaging modalities including magnetic resonance, and radioisotopic imaging. From this concentration data, using the described model, the rate constants can be derived. Using an initial fMRI or other diagnostic procedures (e.g., nuclear medicine) determining the evolution of concentration in each compartment, the exact value of the rate constants can be attained. Having these rate constants, the model can then be solved to produce any desired concentration/steady-state profile.

Therefore, the instant inventors have identified a much more sophisticated intramuscular approach that allows more compartments in this model; the disclosed method allows achieving steady-state DMT concentrations in the target organ/effect site, without having to deal with the venous response. As a corollary, this method includes model delivery concepts: use nano-particles (DMT solute embedded in gelatinous particles) e.g. crystalline freebase DMT particles ranging from nanometer to micrometer sizes, suspended in a viscous or gelatinous medium to shield DMT from the serotonin receptors to allow safer venous delivery. This allows for fewer compartments and is equivalent to setting k₀, k₁ and k₂ equal to zero, and removing the variable [M] from the system.

Currently there is no area within medical practice identified where continuous infusion of a drug into a muscle is used to avoid unnecessary venous cannulation. The only time intramuscular injection is used is for a bolus dose of a medication, or vaccine. It is assumed that there is a maximum upper limit to how much of an injection a muscle can tolerate. Experimental results have demonstrated that venolymphatic flow is sufficient for this use, and is practical for many applications, including DMT infusion.

Intramuscular injection allows for the use of more specially designed needle types. For example, needles may have beveled tip, with a hole at the end, typical of needles used for intramuscular injection. The needle may include a number of side holes ranging from 1-100, more preferably 2-20, which allows for a larger surface area of the infused drug to be taken up and carried away by venous and lymphatic flow. A curve at the base of the needle ranging from 0 to 90 degrees, allows the tubing from an infusion pump to the needle hub to achieve an angle suitable for securing with either tape, an adhesive sterile covering, or the like.

In order to move forward with experiments involving steady states, either an intra-venous method utilizing one or more of the below methods, or infusion via intramuscular route, through the kinetic model derived herein will be required. It is noted that the intravenous infusion can essentially be modeled as a two-compartment transport while intramuscular infusion is modeled as multi-compartment transport. This is because serotonergic compounds freely pass the blood brain barrier transport directly into effector neurons of the CNS while intramuscular injection requires transport through various tissues to reach the effector tissues of the brain.

In this respect, the inventors addressed the following goals:
1. A method for adding low concentration heparin in doses ranging from 0.1-100 IU per mL, more prefer-ably in the range of 1-10 IU per mL, which have been found experimentally to improve flow rate, even though there is negligible risk for an acute thrombus.
2. A method for adding low concentration magnesium in doses ranging from 0.1 mg/mL to 100 mg/mL, more preferably in the range of 1-2 mg/mL, added to either the DMT infusion directly, or to a maintenance line running with normal saline or similar infusate, which have been found experimentally to improve flow rate, and decrease venous injury.
3. A method for combining goals 1 and/or 2 with nitroglycerine ointment on the vein to be cannulated prior to gaining intravenous access, which improves vein patency and dilution from blood flow as well as normal saline.
4. A method for creating an injectable form of a DMT salt such as fumarate, or freebase DMT in a solution or suspension or emulsion in an iso-osmolar or near iso-osmolar carrier such as hyaluronic acid or polyethylene glycol, as to minimize molecular interaction with the walls and endothelium of the peripheral vasculature, while maintaining water soluble or miscible properties as to not adversely affect the pharmacokinetics.
5. A method for utilizing goal 4, with any other drug or substance that may cause venous irritation, to minimize the risk of thrombophlebitis, such as nanoparticles or crystals in suspension, or similarly, chemotherapeutic drugs, anesthetic agents and the like.

5-Meo-DMT (a DMT analog) follows similar kinetics as other serotonergics, and the above methods are presented with adjustments for dose and 5-Meo-DMT specific kinetics.

A method for achieving steady state of an infused drug such as DMT via continuous or intermittent intravenous or intramuscular infusion, via a model based on known parameters of that drug, and feedback of patient experience into this model for additional fine-tuning.

It is also contemplated that topical DMT can be used for “micro-dose” effects.

### Essentials

Until investigators have more clinical data, it is ill-advised to guide a subject through an extended state experience without that subject first having prior experience with either smoked or vaporized DMT. Ayahuasca contains DMT as one of the active ingredients, and while having prior experience with Ayahuasca would be advantageous, however this is insufficient on its own.

The “DMT space” can be overwhelming and difficult to navigate. Overshooting the levels in the CNS can be distressing and even traumatic. The subject's sense of time will usually be altered (dilated). Overshooting this state with intravenous infusion may be distressing until the DMT is either redistributed to another compartment, and or metabolized and/or eliminated. In the case of intravenous infusion, if the initial bolus dose is too high, the experience will be overwhelming and/or distressing, but will rapidly abate due to redistribution of drug into the rest of the body over a steep concentration gradient. Once steady state is achieved, there is no concentration gradient between the CNS and the peripheral tissues. If the dose of DMT is too high once steady state is achieved (roughly 20 minutes after initiation depending on the patient's individual kinetics), it takes much longer for the experience to abate, because redistribution of drug is no longer a mechanism for decreasing CNS levels.

In intramuscular infusion, the muscle is first loaded which then diffuses into the peripheral soft tissues, the lymphatics and blood, and then the CNS. The result is a very gentle rise in effect, with the earliest effects usually noticed within the first 2 minutes, and the subject experiencing threshold effects somewhere between 5 and 15 minutes. The intensification of the experience is slow, and while there is some overshoot, it is quite manageable, and usually lasts for only minutes. The key here is turning the pump off when the ideal state is reached, or at the earliest sign of being above the ideal level and reporting when those levels have been achieved.

This initial model gives significant room for error, allowing for a safe first experience, and collecting individual data such that any subsequent experience can be modeled to a more ideal level, the result being a faster rise in levels to an ideal state, and minimal or no turning off of the pump after initiation. If turning the pump off is required during a subsequent experience (after programmatic modeling with derived rate constants), that data can be entered into the program for a re-calculation of the ideal curves to get an even more ideal fit.

Any concentration of DMT fumarate can be used for this purpose, however 25 mg/ml seems to be preferred as a formula can easily be achieved to be near physiologic pH and iso-osmolar.

The preferred embodiment of the pump involves a syringe pump, programmed to behave in the above-described manner, and then subsequently per the derived infusion rates from the disclosed program, which can give very tight control of the infused amount, and can easily provide enough driving force through the preferred microbore tubing and needle.

As far as needles are concerned, any intramuscular needle is acceptable, however a small gauge needle is more comfortable, and in various embodiments, a 25-gauge needle may be preferred. This can be placed either perpendicular to the skin, or at an angle, however the preferred embodiment involves either the user introducing a bend near the hub of the needle, or a needle manufactured with a bend for this purpose.

A bend of 90 degrees is acceptable so long as the lumen is not narrowed to the point that the driving force required of the pump is problematic. When doing a manual bend, an angle of 30 to 60 degrees is more preferred to avoid this problem. The bend allows for a sterile dressing to be more easily placed over the needle, which helps prevent backing out, during administration of the infusate.

### EXAMPLES

Various exemplary embodiments of devices and compounds as generally described above and methods according to this invention, will be understood more readily by reference to the following examples, which are provided by way of illustration and are not intended to be limiting of the invention in any fashion.

### Example 1

### Two Compartment Modeling: Deficits of the Prior Art

In 2016, Andrew R. Gallimore and Rick J. Strassman published a paper in Frontiers of Pharmacology entitled: A model for the application of target-controlled intravenous infusion for a prolonged immersive DMT psychedelic experience (Article 211, Volume 7, July 2016, hereinafter the “G-S paper” (prior art)). Their final steady-state formula borrows the classical result:

$${k}_{cat} [ ES ] = \frac{{V}_{max} [ S ]}{{K}_{M} + [ S ]}$$

from the Michaelis-Menten (M-M) model, where an enzyme E and a substrate S bind to create an intermediate ES molecule before either returning to E and S or converting to E and P. Here, rate constants kₒₙ, k_{off} and k_{cat} denote these reactions in forward, backward and to E+P directions respectively, K_{M}=(k_{off}+k_{cat})−kₒₙ and Vₘₐₓ=k_{cat} [E]T, and [E]T is the total concentration of the enzyme.

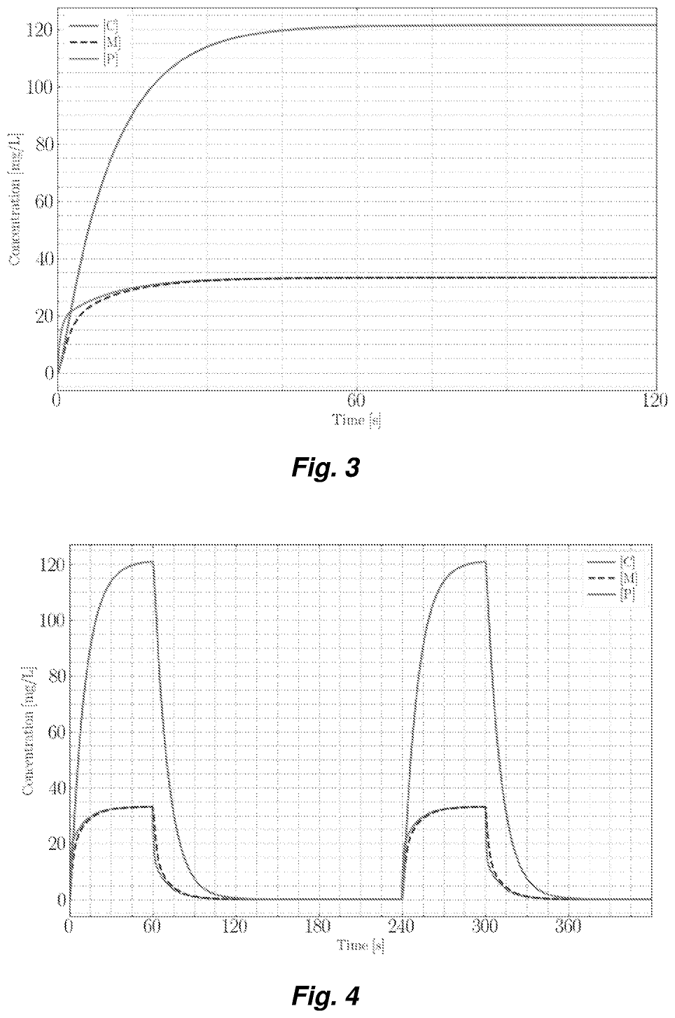

Firstly, those of skill in the art will appreciate that the use of M-M steady-state formula is not applicable to this compartmental model, which involves significantly more variables than appear in the M-M model. Furthermore, the data in this model appears to be completely fabricated. For example, Gallimore and Strassman provide data in Table 2 (G-S paper) that appears to erroneously identify an infusion rate of 0.93 mg/min. Substituting the suggested values in Table 2 into the identified steady-state formula (above) shows that the concentrations are off by at least an order of magnitude. To rule out typographical errors, setting up an excel spreadsheet to calculate the steady state elimination rate (R_{SS}) and varying the entries, shows that this formula (as expected) is quite stable to changes in the parameter, and the value of 0.93 mg/min cannot be achieved without using ridiculously large entries. Indeed, FIG. 2 of the G-S paper purports to fit the clearance of drug to each of the concentrations 0.4 and 0.2 mg/kg used but illustrates when administered the higher dose (FIG. 2A the patient achieved only ⅓ the blood concentration (100 ng/ml) than those receiving the lower dose (300 ng/kg). Thus, clearly the G-S model is in error.

Instead of trying to back-solve Gallimore and Strassman (G-S) results and try to find out how they arrive at their steady-state rates and concentrations, here constructed is a two-compartment model from scratch and solve this problem. Some of the methods used here will be the same as those developed in more detail for a more general model in the sections below. This model may have its own intrinsic value, in dealing with either different substances, or if an appropriate mechanism is found to deliver DMT intravenously, for this drug.

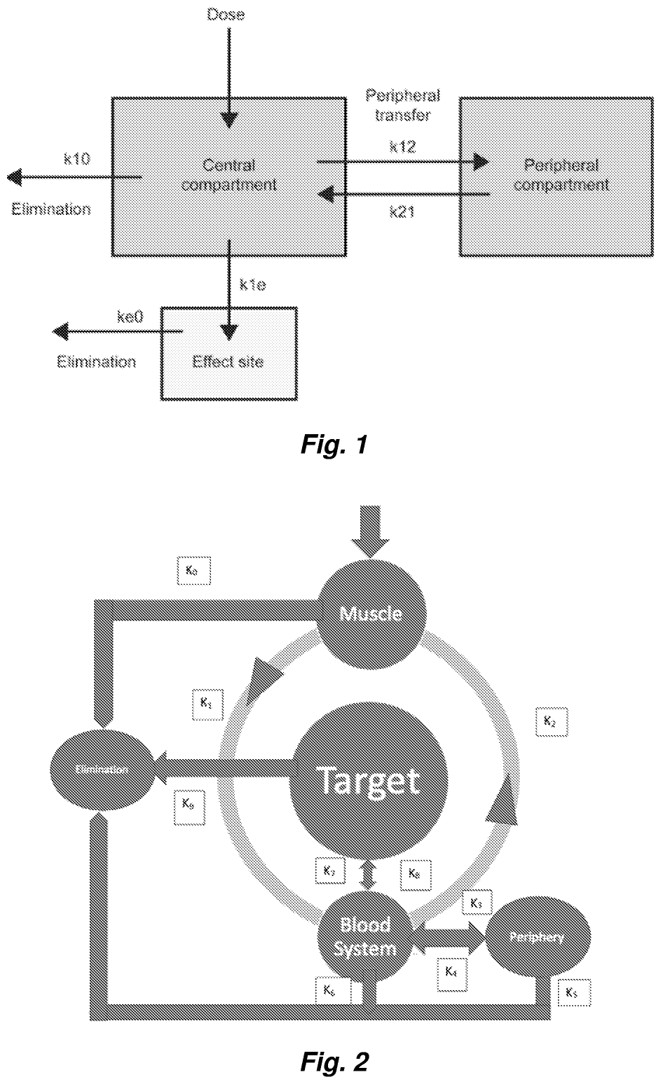

For consistency with the G-S model, referred to here as FIG. 1 is the (prior art) model from the 2016 paper by Gallimore Strassman (prior art), where:
k₁₀ rate of elimination from the central compartment (min⁻¹)
k₁₂ transfer rate from central to peripheral compartment (min⁻¹)
T same as k₁₂
k₂₁ transfer rate from peripheral to central compartment (min⁻¹)
K₁ₑ transfer rate from central compartment to effect site (min⁻¹)
Kₑ₀ rate of elimination of drug from the effect site (min⁻¹) E same as kₑ₀
C_{T} plasma concentration (mg/ml)
R_{T} rate at which drug reaches the effect site (mg/min)
[C] DMT concentration in the central compartment (mg/ml)
[P] DMT concentration in the peripheral compartment (mg/ml) concentration in the effect site (mg/ml).
[M] DMT concentration in the effect site (mg/ml).

It should be appreciated that the notation used in the prior art (G-S) is very different from the notation used herein in developing the models in the present disclosure. In G-S (p. 5), E is used for the elimination rate, without it being made clear if this is the elimination rate from the effect site or from the central compartment. Also, T is used interchangeably with k₁₂ from G-S FIG. 1. The extra parameters in G-S Table 2, V_{c} (L), Vₚ (L), Kₘ (mg/L), Vₘ (mg/min), Q₁₂ (L/min) are not defined, modulo a minor footnote (bottom of p. 4) stating that:

$${κ}_{12} = {Q}_{12} / {V}_{c} ; and {κ}_{21} = {Q}_{12} / {V}_{p}$$

Using these relations gives:

| Concentration (mg/kg) | k₁₂ (min⁻¹) | k₂₁ (min⁻¹) |
|---|---|---|
| 0.4 | 0.60215 | 0.16470 |
| 0.2 | 0.32857 | 0.20000 |

In the G-S model it is hard to understand how a DMT bolus of 0.2 mg/kg (FIG. 2(B) [G-S]) produces an almost 3 times greater concentration than a DMT bolus of 0.4 mg/kg (FIG. 2 (A) [G-S]).

Starting from basic principles, the system of differential equations guiding the change of concentration in this 2-compartment model is:

$$d [ C ] / dt = - {k}_{12} [ C ] + {k}_{21} [ P ] - {k}_{10} [ C ] - {k}_{{1}_{e}} [ C ] + f ( t ) \qquad ( 2 )$$

$$d [ M ] / dt = {k}_{{1}_{e}} [ C ] - {k}_{{e}^{0}} [ M ]$$

$$d [ P ] / dt = {k}_{12} [ C ] - {k}_{21} [ P ]$$

along with the initial conditions:

$$[ C ] ( 0 ) = [ M ] ( 0 ) = [ P ] ( 0 ) = 0.$$

Denoting by x the vector ([C]; [M]; [P])′ with prime denoting the transpose of the given vector, this system of differential equations can be written in vector form as:

$$dx / dt = Ax + F \\ where : \\ A = [ \begin{matrix}- ( {k}_{12} + {k}_{10} + {k}_{1 e} ) & 0 & {k}_{21} \\ {k}_{1 e} & - {k}_{e 0} & 0 \\ {k}_{12} & 0 & - {k}_{21}\end{matrix} ]$$

and F=(ƒ(t), 0, 0)′ is the pump infusion protocol.

The eigenvalues of the matrix A can be determined readily. These are:

$$\begin{matrix}{λ}_{1} = - {k}_{e 0} \\ {λ}_{2 , 3} = \frac{1}{2} ( - R \pm \sqrt{{R}^{2} - 4 ( {k}_{10} {k}_{21} + {k}_{1 e} {k}_{21} )}\end{matrix}$$

where R=k₁₂+k₂₁+k₁₀+K₁ₑ. Since all the rate constants are nonnegative, it can be easily shown that all the eigenvalues are negative, thus leading to an asymptotically stable outcome. Solving for the eigenvectors associated with these eigenvalues, obtained is ξ₁=(0, 1, 0)′ and ξ_{2, 3} are fairly long expressions given by

$$α = {k}_{12} + {k}_{10} + {k}_{1 e} - {k}_{21} \\ β = \sqrt{{( {k}_{12} + {k}_{10} )}^{2} + {( {k}_{1 e} - {k}_{21} )}^{2} + 2 ( {k}_{1 e} ( {k}_{12} + {k}_{10} ) + {k}_{21} ( {k}_{12} - {k}_{10} ) )} \\ {ξ}_{2} = {( - \frac{α + β}{2 {k}_{12}} , - \frac{- {k}_{1 e} ( α + β )}{{k}_{12} ( R + β - 2 {k}_{e 0} )} , 1 )}^{′} \\ and \\ {ξ}_{3} = {( - \frac{α - β}{2 {k}_{12}} , - \frac{{k}_{1 e} ( α - β )}{{k}_{12} ( - R + β + 2 {k}_{e 0} )} , 1 )}^{′} .$$

Mathematica has been used to verify these eigenvectors. ξ₂ is the eigenvector corresponding to the minus sign in ∵ in the eigenvalues, and ξ₃ is that for the positive sign. Now the solution of this 2-compartment model can be written as:

$${y}_{j} ( t ) = {e}^{{λ}_{j} t} {\int }_{0}^{t} {e}^{- {λ}_{j} s} {h}_{j} ( s ) ds + {c}_{j} {e}^{{λ}_{j} t} , j = 1 , 2 , 3 \qquad ( 3 )$$

where Y(t)=(y₁(t), y₂(t), y₃(t))=\hat{T}⁻¹X(t), H(t)=(h₁(t), h₂(t), h₃(t))=\hat{T}⁻¹F(t), and \hat{T}₁s the matrix of eigenvectors of Â. Having the explicit form of the eigenpairs of this system, the solution Y(t) and X(t) can be written down. Plugging in the empirically derived rate constant in the G-S model, and the initial bolus regimens followed by a steady infusion rate, now provided is a graphical display of approach to steady state for this model. Used is:

F=(0.4,0,0)′,k₁₂=0.6025,k₂₁=0.16470.

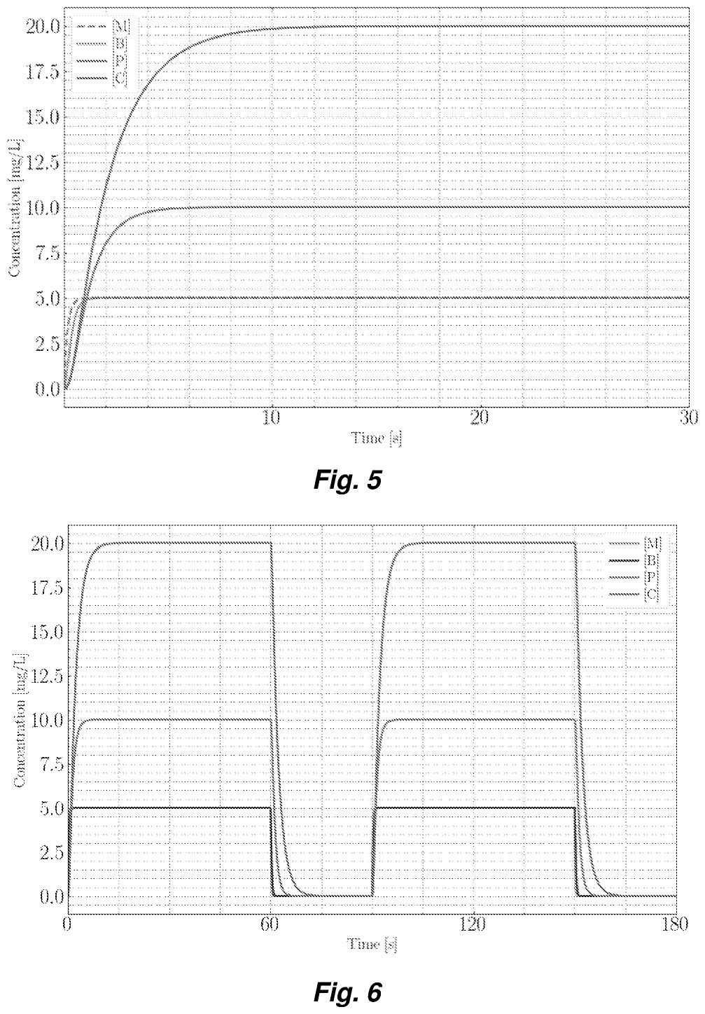

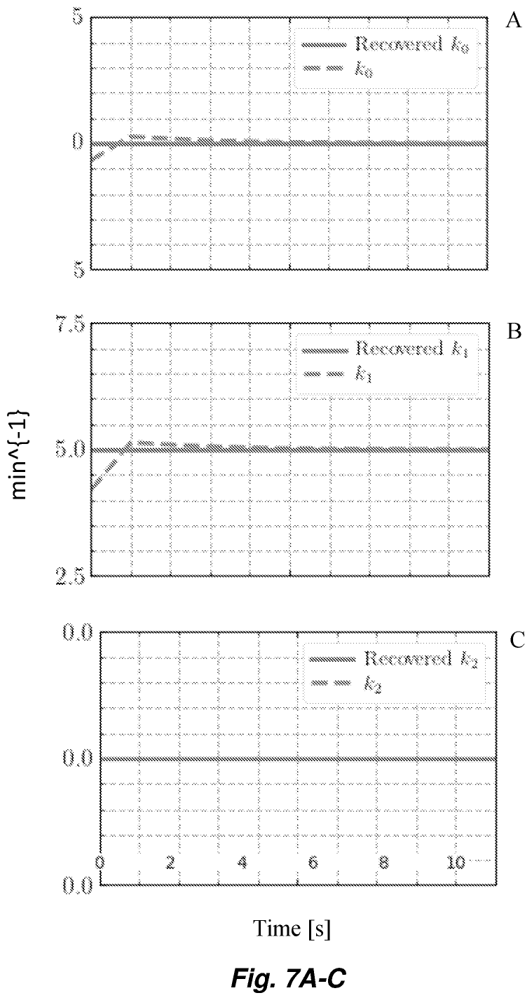

The rate constants k₁₀, k₁ₑ and kₑ₀ are not specified in the G-S paper. Let's assume that close to equilibrium k₁ₑ≈kₑ₀, so k₁ₑ=kₑ₀=k₁₂=0.6025 and k₁₀=0.3012 (set arbitrarily). Consider two cases: (1) a steady infusion regimen of 0.4 mg/kg/min, and (2) an initial large bolus of 30 mg (0.4×75=30 mg for a 75 kg man), a transient wait time of 3 min, followed by a steady infusion of 0.4 (mg/kg)/min. FIGS. 2 and 3 describe approach to equilibrium of the DMT concentration for this model in each of the compartments. It is noteworthy that the DMT concentration in the blood and effect site have a very rapid rise (FIG. 3), and reaches a steady-state concentration of approximately 32-33 mg/ml. In FIG. 4, under an on/off infusion regimen, a similar rise is seen followed by very rapid dissipation of the concentration levels in the next 60 sec. This is consistent with the acquired by the inventors. The rate constants used here are those given by the G-S paper and some assumptions are made for the rate constants that are not specified. This thorough analysis of the 2-compartment model justifies a refute of the G-S results.

A second, and perhaps more serious issue of the G-S two compartment model is the suggested administration of DMT intravenously. Based on data acquired by the inventors, it appears that the serotonin receptors in the venous walls interact with DMT, causing vasoconstriction, phlebitis and eventual closing of the infusion veins, and possibly resulting in permanent venous injury. This reaction introduces a significant concentration dependence of the rate constants at the infusion site, affecting the transport of the drug to the bloodstream and makes the model significantly more complicated, if not completely inapplicable or clinically hazardous.

The deficits of the G-S model required the development of a more sophisticated intramuscular approach and allowing more compartments in this model, this method will allow achieving steady-state DMT concentrations in the target organ/effect site, without regard to the venous response. As a corollary, in this disclosure will also extend the present model to include delivery concepts: e.g. Crystalline freebase DMT particles ranging from nanometer to micrometer sizes, suspended in a viscous or gelatinous medium to shield DMT from the serotonin receptors to allow venous delivery. This case will lead to fewer compartments, which would be equivalent to setting k₀, k₁ and k₂ equal to zero, and removing the variable [M] from the system.

### Example 2

### Multi-Compartment Modeling

In this model, the concentration of the drug in the muscle, blood stream, peripheral organs and the target organ/effect site by [M], [B], [P] and [C] respectively are denoted. With reference to FIG. 4, the complexity of the problem is illustrated. In the general case, the rate constants “k” are functions of the concentration gradients along the transport path. Used herein are:
k₀ rate of elimination of the drug from the muscle;
k₁ rate of transport of the drug from the muscle into the blood system;
k₂ rate of transport of the drug from the blood stream back into the same muscle;
k₃ rate of transport of the drug from the blood stream to the peripheral organs/muscles;
k₄ rate of transport of the drug from the peripheral organs/muscles back to the blood stream;
k₅ rate of elimination of the drug from the peripheral organs/muscles;
k₆ rate of elimination of the drug from the blood stream;
k₇ rate of transport of the drug from the blood stream to the target organ;
k₈ rate of transport of the drug from the target organ back to the blood stream;
k₉ rate of elimination of the drug from the target organ.

It is worthwhile to note that elimination of the drug from each compartment is intended to include all processes such as elimination, metabolism, inactivation and other chemical and nuclear (in case of radioisotopes) reactions leading to the breakdown of the drug under consideration. Also, the transport of the drug into the muscle, from the muscle to the bloodstream, and from the bloodstream to the peripheral organs and to the target organ, is guided by Newton's first law of transport that states that the rate of transfer is proportional to the concentration gradient of the diffusing substance, i.e. k∝d[·]/ds where k is the rate of transfer, [·] is the concentration of the diffusing substance and s is the arc length along the travel path. This clearly implies that the rate of transfer between the various compartments is related to the concentration in those compartments, thus making the system of differential equations highly nonlinear. Two separate cases arise here:
1. Cases where the drug action, metabolism and inactivation is fast relative to transport times. In this case, the concentrations are not allowed to build to such an extent to change the transport time constants appreciably, and the rate constants can be assumed constant.
2. Cases where the drug action, metabolism and inactivation is significantly slower than transport times, where the buildup of the drug concentration in each organ is sufficient to change the time constants significantly. Here, the rate constants will not be constants and the fully nonlinear problem must be considered to arrive at a predictive model.

$$k = constant \times ( {[ \cdot ]}_{2} - {[ \cdot ]}_{1} ) . \qquad ( 1 )$$

For example, k₁(t)=α₁([B](t)−[M](t))=−k₂(t), k₃(t)=−k₄(t)=α₄([P](t)−[B](t)) and so on, where α1 and α4 are proportionality constants. Using directional dependence of most of these transport processes, additional proportionality constants, such as k₁(t)=−γk₂(t), can be introduced as well. Both cases are fully solved here and develop a thorough qualitative assessment of each of the cases.

This constructed model provides the full temporal variation of concentration in each of the organs under arbitrarily programmed infusion rates and performs an analysis of the asymptotic behavior of the drug concentration and approach to steady-state. It also provides a study of infusion models and parameter ranges, giving ideal approaches to steady state without significant overshoot or lengthy buildup. One surprising and novel component of this model is the ability to determine the rate constants (both constant and time dependent) in closed form from experimental (diagnostic) measurement of concentrations as a function of time in a preliminary run. Equations 18 and 19 together provide those rate constants, once the concentrations are measured. Hence there is no need to appeal to curve-fitting or other forms of approximation as needed in the G-S model. Having these rate constants, this model will provide a state-of-the-art tool for drug delivery including both intravenous and intramuscular routes of administration.

The models disclosed herein achieve these rate constants from much simpler and more straightforward measurements. While the model may not give the exact membrane transport mechanisms which are the basis for these rate constants, it does achieves the eventual goal that will be crucial for developing effective treatment planning and drug delivery.

The following work provides a highly robust compartmental model allowing us to answer the following key questions:
1. Given the rate constants k₀; _ _ _ ; k₉ and a pre-scribed infusion function ƒ(t), determine [C](t) in the target organ? What is the long time behavior of [C](t)?
2. Which ƒ(f) gives an optimal approach to steady-state concentration [C]_{[S]} in the target organ?
3. Can one determine the rate constants from the dynamics of the concentrations in the different compartments?
4. How much of the above analysis can be carried out in its full generality in the nonlinear cases, where the rate constants satisfy Eq 1 or other nonlinear processes?

### Example 3

### Kinetic Model of Drug Transport: Case 1

The kinetics of the model in FIG. 2 may be described by the following system of differential equations:

$$\begin{matrix}\frac{d [ M ]}{dt} = - ( {k}_{0} + {k}_{1} ) [ M ] + {k}_{2} [ B ] + f ( t ) \\ \frac{d [ B ]}{dt} = {k}_{1} [ M ] - ( {k}_{2} + {k}_{3} + {k}_{6} + {k}_{7} ) [ B ] + {k}_{4} [ P ] + {k}_{8} [ C ] \\ \frac{d [ P ]}{dt} = {k}_{3} [ B ] - {k}_{4} [ P ] - {k}_{5} [ P ] = {k}_{3} [ B ] - ( {k}_{4} + {k}_{5} ) [ P ] \\ \frac{d [ C ]}{dt} = {k}_{7} [ B ] - {k}_{8} [ C ] - {k}_{9} [ C ] = {k}_{7} [ B ] - ( {k}_{8} + {k}_{9} ) [ C ]\end{matrix} \qquad ( 4 )$$

along with a set of appropriate initial conditions

$$[ M ] ( 0 ) = [ B ] ( 0 ) = [ P ] ( 0 ) = [ C ] ( 0 ) = 0$$

To determine the steady-state concentration [C](t), set are d[C]/d=0. Providing:

$${k}_{7} [ B ] = ( {k}_{8} + {k}_{9} ) [ C ] .$$

This is the consistency relation that must be satisfied at steady-state. If at steady-state the concentration [C] is [C]_{[S]}, this provides:

$${[ C ]}_{s} = \frac{{k}_{7}}{{k}_{8} + {k}_{9}}$$

In vector form, the system of Eq. 4 take a form more suitable for analysis. Letting X=([M], [B], [P], [C])′, F(t)=(ƒ(t), 0, 0, 0)′, where ′ denotes the transpose, and

$$\hat{A} := ( \begin{matrix}- ( {k}_{0} + {k}_{1} ) & {k}_{2} & 0 & 0 \\ {k}_{1} & - ( {k}_{2} + {k}_{3} + {k}_{6} + {k}_{7} ) & {k}_{4} & {k}_{8} \\ 0 & {k}_{3} & - ( {k}_{4} + {k}_{5} ) & 0 \\ 0 & {k}_{7} & 0 & - ( {k}_{8} + {k}_{9} )\end{matrix} ) \qquad ( 5 )$$

then the system of equations (2) can be written in compact form as

$$\frac{dX}{dt} = \hat{A} X + F , X ( 0 ) = 0. \qquad ( 6 )$$

Using classical methods to solve the non-homogeneous system of differential equations in (6), if the eigenpairs of the matrix Â are denoted by (λᵢ, ξᵢ), i=1, 2, 3, 4, the solutions of the system (6) are expressed by:

$${y}_{j} ( t ) = {e}^{{λ}_{j} t} {\int }_{0}^{t} {e}^{- {λ}_{j} s} {h}_{j} ( s ) ds + {c}_{j} {e}^{{λ}_{j} t} , j = 1 , 2 , 3 , 4 \qquad ( 7 )$$

where Y(t)=(y₁(t), y₂(t), y₃(t), y₄(t))=\hat{T}⁻¹X(t), H(t)=(h₁(t), h₂(t), h₃(t), h₄(t))=\hat{T}⁻¹F(t), and \hat{T} is the matrix of eigenvectors of λ.

### Explicit Solution and Asymptotic Behavior

To arrive at asymptotic behavior of the concentration of the drug in the different compartments of this model, and more specifically in the target organ/effect site, let us note that the determinant of Â can be readily written as:

$$det \hat{A} = αβδγ - αδ {k}_{3} {k}_{4} - αγ {k}_{7} {k}_{8} - γδ {k}_{1} {k}_{2} , \qquad ( 8 )$$

where α, β, γ and δ are the absolute values of the diagonal entries of the matrix Â i.e. the cumulative and nonnegative rate constant of the drug leaving each compartment. It is interesting to observe how this determinant depends on the pairwise products k₁k₂, k₃k₄ and k₇k₈. These are the rate constants in and out of each of the compartments M, B and C. So, if any of the rate constants in each of these products is set equal to zero, the effect of that compartment on the determinant disappears.

Itis important to ensure that the matrix A does not become singular. To see this, note that det Â=0 if:

$$αβδγ = αδ {k}_{3} {k}_{4} + αγ {k}_{7} {k}_{8} + γδ {k}_{1} {k}_{2} . \qquad ( 9 )$$

However, the term on the left-hand side has 32 nonnegative terms, and each of the terms on the right hand side of this equation consists of a subset of 4 of the same terms in the left hand side of this equation. Since each of the terms here are non-negative, the equality in Eq. (9) is never achieved (in general). Hence the matrix Â is nonsingular. Therefore, the matrix of eigenvectors of Â, \hat{T}, is full rank and invertible, as it is necessary for the solution expressed in (4) to hold. With the calculation of eigenvalues and eigenvectors of the matrix Â, and Y given by Eq (7), provides:

$$X ( t ) = \hat{T} Y ( t ) . \qquad ( 10 )$$

To determine the eigenvalues of the matrix Â explicitly, the quartic equation (in the variable λ) is solved by:

$$\begin{matrix}0 = ( α - λ ) ( β - λ ) ( δ - λ ) ( γ - λ ) \\ - ( α - λ ) ( δ - λ ) {k}_{3} {k}_{4} \\ - ( α - λ ) ( γ - λ ) {k}_{7} {k}_{8} \\ - ( γ - λ ) ( δ - λ ) {k}_{1} {k}_{2}\end{matrix} \qquad ( 11 )$$

While an analytical solution of this general quartic equation is at best tedious or, possibly by Galois theory, may not have an algebraic expression over R in general, the inventors determined the asymptotic behavior of the solution based on numerical approximation of eigenvalues (and eigenvectors).

### Example 4

For illustrative purposes, let us consider an important example to emphasize the analysis and approach to steady-state solutions.

$$Assume : {k}_{2} = 0 , {k}_{4} = 0 , {k}_{8} = 0$$

The selection of these parameters is justified by suggesting that the concentration of the drug in the infusion muscle is always higher than the blood stream, so by Newton's law the drug does not diffuse back into that muscle against a concentration gradient. Also, by the same reasoning, k₄ and k₈ may be assumed to be zero. Then the matrix Â becomes:

$$\hat{A} = ( \begin{matrix}- ( {k}_{0} + {k}_{1} ) & 0 & 0 & 0 \\ {k}_{1} & - ( {k}_{3} + {k}_{6} + {k}_{7} ) & 0 & 0 \\ 0 & {k}_{3} & - {k}_{5} & 0 \\ 0 & {k}_{7} & 0 & - {k}_{9}\end{matrix} ) \qquad ( 12 )$$

Since this is lower triangular, the eigenvalues are the diagonal entries. That is:

$$\begin{matrix}{λ}_{1} = - ( {k}_{0} + {k}_{1} ) \\ {λ}_{2} = - ( {k}_{3} + {k}_{6} + {k}_{7} ) \\ {λ}_{3} = - {k}_{5} \\ {λ}_{4} = - {k}_{9}\end{matrix} \qquad ( 13 )$$

and the corresponding eigenvectors are:

$$\begin{matrix}{\vec{ξ}}_{1} = {( 1 , \frac{{k}_{1}}{{λ}_{1} - {λ}_{2}} , \frac{{k}_{1} {k}_{3}}{( {λ}_{1} - {λ}_{2} ) ( {λ}_{1} - {λ}_{2} )} , \frac{{k}_{1} {k}_{7}}{( {λ}_{1} - {λ}_{2} ) ( {λ}_{1} - {λ}_{4} )} )}^{′} \\ {\vec{ξ}}_{2} = {( 0 , 1 , \frac{{k}_{3}}{{λ}_{2} - {λ}_{3}} , \frac{{k}_{7}}{{λ}_{2} - {λ}_{4}} )}^{′} \\ {\vec{ξ}}_{3} = {( 0 , 0 , 1 , 0 )}^{′} \\ {\vec{ξ}}_{4} = {( 0 , 0 , 0 , 1 )}^{′} .\end{matrix} \qquad ( 14 )$$

In the above it is assumed that λᵢ are distinct and nonzero, that's λ₁≠λᵢ, i=2, 3, 4 and λ₂≠λⱼ, j=3, 4. If λ₃=λ₄, then this is an eigenvalue of algebraic multiplicity 2. However, it can be readily seen that the eigenvectors associated with these multiple eigenvalues have full geometric multiplicity, and the matrix A remains invertible, provided these eigenvalues are nonzero. If λ₁ or λ₂=0, non-negativity of the rate constants implies k₀=k₁=0 or k₃=k₆=k₇=0, which of course leads to [C](t)=0, thus the entire model is trivialized.

Hence the homogeneous solution of the system of equations (2) is given by

$${X}_{h} = {c}_{1} {e}^{- {λ}_{1} t} {ξ}_{1} + {c}_{2} {e}^{- {λ}_{2} t} {ξ}_{2} + {c}_{3} {e}^{- {λ}_{3} t} {ξ}_{3} + {c}_{4} {e}^{- {λ}_{4} t} {ξ}_{4} . \qquad ( 15 )$$

$$Since$$

$$\hat{T} = ( \begin{matrix}1 & 0 & 0 & 0 \\ \frac{{k}_{1}}{{λ}_{1} - {λ}_{2}} & 1 & 0 & 0 \\ \frac{{k}_{1} {k}_{3}}{( {λ}_{1} - {λ}_{2} ) ( {λ}_{1} - {λ}_{3} )} & \frac{{k}_{3}}{{λ}_{2} - {λ}_{3}} & 1 & 0 \\ \frac{{k}_{1} {k}_{7}}{( {λ}_{1} - {λ}_{2} ) ( {λ}_{1} - {λ}_{4} )} & \frac{{k}_{7}}{{λ}_{2} - {λ}_{4}} & 0 & 1\end{matrix} ) ,$$

Noting that \hat{T} is an elementary matrix, Gauss-Jordan algorithm may be used to get:

$${\hat{T}}^{- 1} = ( \begin{matrix}1 & 0 & 0 & 0 \\ - \frac{{k}_{1}}{{λ}_{1} - {λ}_{2}} & 1 & 0 & 0 \\ \frac{{k}_{1} {k}_{3}}{( {λ}_{1} - {λ}_{3} ) ( {λ}_{2} - {λ}_{3} )} & \frac{{k}_{3}}{{λ}_{2} - {λ}_{3}} & 1 & 0 \\ \frac{{k}_{1} {k}_{7}}{( {λ}_{1} - {λ}_{4} ) ( {λ}_{2} - {λ}_{4} )} & \frac{{k}_{7}}{{λ}_{2} - {λ}_{4}} & 0 & 1\end{matrix} ) \qquad ( 16 )$$

Using Eq. 7 followed by Eq. 10, provides X(t).

To achieve a better understanding of the asymptotic behavior of the concentration of the substance in the target organ, it is important to return to Eq. (7) and Eq. (10) in the above model. Under the most typical initial conditions of X(0)=0, the coefficients of the homogeneous equation are all zero, and thus the drug concentration is guided by a linear superposition:

$$X ( t ) = \hat{T} ( {e}^{Λ t} * {\hat{T}}^{- 1} F ( t ) ) \qquad ( 17 )$$

where * denotes the convolution from 0 to t, and Λ is a diagonal matrix with the entries of eigenvalues of Â. It is easy to note that for a given desired time evolution of the drug in the different organs X(t), Eq. (17) can be solved for F(t) to give

$$F ( t ) = \hat{T} {ℱ}^{- 1} ( {( ℱ {e}^{Λ t} )}^{- 1} ℱ {\hat{T}}^{- 1} X ( t ) ) \qquad ( 18 )$$

[symbol]

denotes the Fourier transform in the above formula. It is quite interesting that not only the asymptotic concentration can be prescribed here, but the full evolution of the drug in any of the compartmental organs can be prescribed by programming F(t) as suggested by Eq. (18).

The problem however becomes somewhat more restrictive if F(t) is required to be of the form (ƒ(t), 0, 0, 0)′. That is the drug is only introduced intramuscularly, and ƒ(t) is the prescribed drug delivery regimen. In this situation, Eq. (17) can be simplified in the form

$$X ( t ) = \hat{T} ( {e}^{Λ t} * {\hat{T}}^{- 1} F ( t ) ) = \hat{T} {e}^{Λ t} {\int }_{0}^{t} {e}^{- Λ s} {{\hat{T}}^{- 1} ( f ( s ) , \vec{0} )}^{′} ds \qquad ( 11 )$$

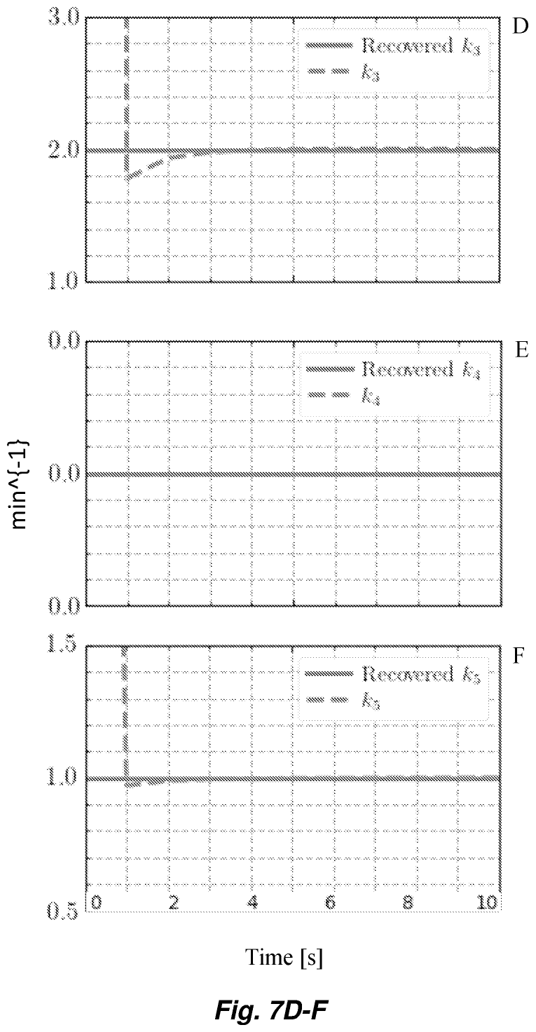

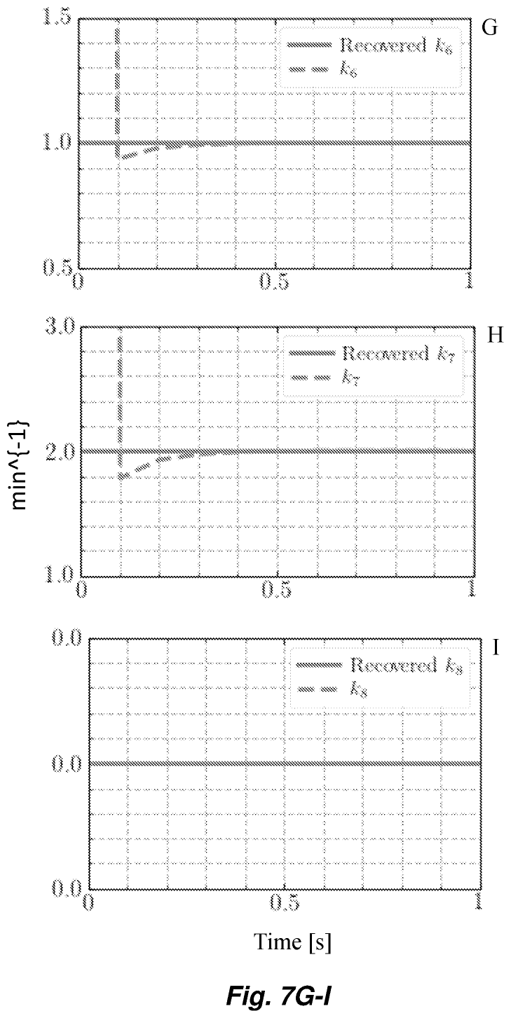

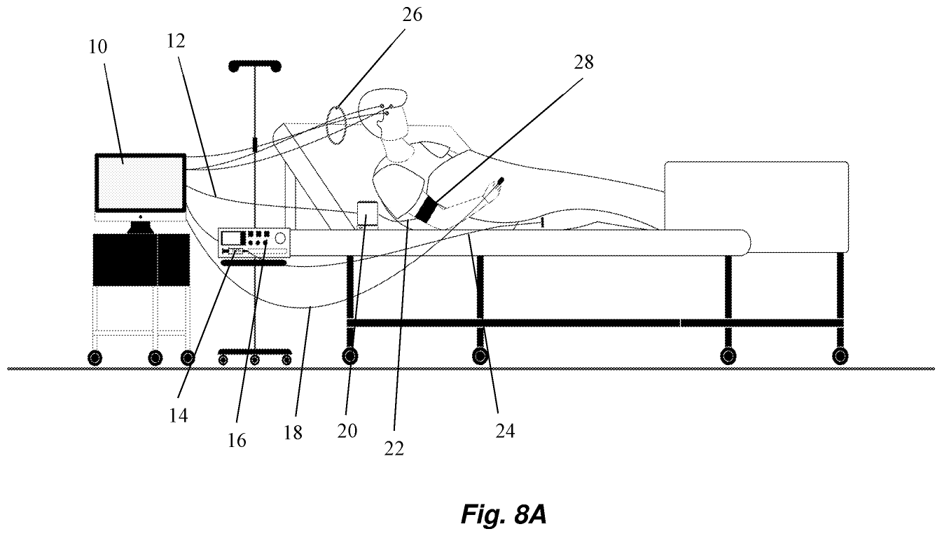

The integral of a vector function is, as usual, the term by term integral of that expression. As t→∞, the eigenvalue with the smallest absolute value will determine the asymptotic behavior of the concentration in the target organ. FIGS. 5 and 6 display the evolutions of concentrations in various compartments in the 5-compartment model for infusion into muscle. FIG. 5 uses rate constants k0; _ _ _ ; k9 given by [0.1; 0.5; 0.2; 0.1; 0.1; 0.2; 0.05] respectively, initial conditions of 0 for all the concentrations, and a constant forcing function and FIG. 7 shows the values of the rate constants extracted from the concentrations in the 4 compartments, as displayed in FIGS. 5 and 6. Convergence to the rate constants used in those earlier figures are displayed in these FIGS. 7A-7H. It is significant to see the rapid convergence to the same rate constants used in the 5-compartment model. The very rapid decay of the DMT concentrations was reflective of data collected and disclosed herein. However, it should be noted that k0; _ _ _ ; k9 were somewhat arbitrarily chosen, and no attempt was made to derive these rate constants based on the methods described below.

### Extraction of the Rate Constants

Perhaps the most serious and significant issue in the above linear model is the rate constants. For the above model to lead to clinically useful outcomes, the rate constants must be determined with some degree of accuracy. Of course, some measurements will be necessary to find these constants. This problem is treated both analytically and numerically in this section.

As earlier, letting X(t) denote the vector of the concentrations as a function of time, and introducing a new vector K=(k0; k1; _ _ _ ; k9)′, the system of equations in (4) can be written as:

$$\frac{dX}{dt} = \hat{D} K + F$$

Note that the column sums of many of the columns in \hat{D} are zero, except for the columns associated with the elimination rate constants. Multiplying both sides of this equation by \hat{D}′, provides:

$${\hat{D}}^{′} \frac{dX}{dt} \text{=\textbar} ( {\hat{D}}^{′} \hat{D} ) K + {\hat{D}}^{′} F \qquad ( 19 )$$

and using the Moore-Penrose pseudo inverse of \hat{D}, provides:

$$K = {( {\hat{D}}^{′} \hat{D} )}^{- 1} {\hat{D}}^{′} [ \frac{dX}{dt} - F ] . \qquad ( 20 )$$

The K determined in this manner is the least-square error solution that provides the rate constants from experimental/clinical measurements of the concentrations X and dX/dt.

Since k0; k5; k6 and k9 are proportional to concentrations [M]; [P]; [B] and [C] respectively, these values of k span the space and can determine the rest of the k values. Consequently, \hat{D} is found to be a 4×4 matrix determined by the linearly independent rate constants and is given by

$$\hat{𝒟} = ( \begin{matrix}\frac{( {α}_{1} - {α}_{0} ) [ M ] + {α}_{2} [ B ]}{{α}_{0}} & 0 & - \frac{{α}_{1} [ M ] + {α}_{2} [ B ]}{{α}_{6}} & 0 \\ - \frac{{α}_{1} [ M ] + {α}_{2} [ B ]}{{α}_{0}} & - \frac{{α}_{a} [ B ] + {α}_{4} [ P ]}{{α}_{5}} & \frac{{α}_{1} [ M ] + ( {α}_{2} + {α}_{3} - {α}_{6} + {α}_{7} ) [ B ] + {α}_{4} [ P ] + {α}_{8} [ C ]}{{α}_{6}} & - \frac{{α}_{7} [ B ] + {α}_{8} [ C ]}{{α}_{9}} \\ 0 & \frac{{α}_{a} [ B ] + ( {α}_{4} - {α}_{5} ) [ P ]}{{α}_{5}} & \frac{{α}_{a} [ B ] + {α}_{4} [ P ]}{{α}_{6}} & 0 \\ 0 & 0 & - \frac{{α}_{7} [ B ] + {α}_{8} [ C ]}{{α}_{6}} & \frac{{α}_{7} [ B ] + ( {α}_{8} - {α}_{9} ) [ C ]}{{α}_{9}}\end{matrix} ) . \qquad ( 21 )$$

Now using Eq. 20, namely K=(\hat{D}′\hat{D})⁻¹\hat{D}′ [dX/dt−F], at each time step, will give us the four independent values of k as a function of time (k's are constant in this case; this is an estimate of the k's as a function of time). These four values are used, the α's, and X(t) to construct the rest. FIGS. 7A, B, C, D, E, F, G, and H shows the values of the rate constants extracted from the data. Given 4 time dependent concentrations as plotted in FIGS. 5 and 6, rearrangement of these concentrations, allow us to establish 4 independent rate constants, k(t) and treating the remaining 5 rate constants as dependent variables. Backward solving for these rate constants, produces the same rate constants used in the 5-compartment model (FIGS. 5 and 6).

Use of the actual rate constants obtained in this way (from the initially measured concentrations) will facilitate control of the prescribed concentrations in a clinical setting. The time dependent rate constants here also account for concentration-dependent rate constants (Newton's model) and possibly rate constants depending on more complex interactions in and between the different model compartments. It is important to point out that once the rate constants are determined, the model described by the system of coupled differential equations in (4) can be executed without a need to re-determine those constants. The extent to which these rate constants are subject dependent and how they vary as a function of time and concentrations can be subjects for many clinical studies. Variations in rate constants may also signal physiologic abnormalities and may be foundational in the development of significant diagnostic tools.

### Example 5

### Pre-Experience Counseling

Prior to initiating an extended or steady state experience with any serotonergic or other psychedelic, there are several considerations to be considered. First and foremost, what is to be accomplished during the experience should be discussed ahead of time, in what will be described here as a pre-psychedelic counseling session or sessions. For a subject engaging in psychedelic therapy, these pre-psychedelic counseling sessions will focus on specific subject matter that the individual is working on, to enable this material to be in the forefront of the subject's mind. In the case of a subject who is a participant in a scientific study that is not focused on therapy, this pre-psychedelic counseling may still provide the individual to recognize the possibility of material of a personal nature arising during the psychedelic experience.

In both the case of therapy, and in the case of scientific research, time should be spent during the pre-psychedelic counseling session to prepare for the experience. In this example, it is assumed that a subject has prior experience with psychedelic compounds, however recognize that there may be scenarios where this isn't the case. Setting expectations with a psychedelic naive subject will be difficult, and it is important to perform a psychological evaluation by a qualified professional to determine that the subject is appropriate for such an experience.

Furthermore, a full physical exam should be provided prior to administration of any psychedelic drug. While many psychedelic drugs have a favorable physiological safety profile, specific drugs such as 5-meo-dimethyltryptamine and ibogaine can have undesirable cardiovascular effects, and caution is required with patients who have comorbidities or underlying medical conditions.

Once the pre-psychedelic counseling session(s) have been completed, as well as an appropriate physical exam has been obtained, the subject's vital signs including blood pressure, temperature and heart rate, should be taken on the day of the psychedelic experience to ensure that there has been no acute change in health status that might warrant re-evaluation.

Once the pre-psychedelic counseling session(s) have been completed, as well as an appropriate physical exam has been obtained, the subject's vital signs including blood pressure, temperature and heart rate, should be taken on the day of the psychedelic experience to ensure that there has been no acute change in health status that might warrant re-evaluation.

Furthermore, exclusion criteria and contraindications should be considered, such as personal or family history of psychiatric illness such as schizophrenia. Psychiatric illnesses such as schizophrenia need to be further studied with respect to psychedelic therapy, and the safety and efficacy of psychedelic drugs in these patient populations is not fully known.

Pre-psychedelic counseling should also include a good personal history, including drug use. This is important to determine a person's prior experience with psychedelic drugs, as well as to understand any history of drug or alcohol abuse, as addiction is one of the indications believed to be a potential clinical target of psychedelic therapy. If a subject has prior DMT experience, the preferred dose that they require to have a comfortable experience should be recorded. If a subject routinely smokes 50 mg of DMT, this will be referred to as a “smoked equivalent.” Vaporizing DMT tends to be more efficient that smoking DMT in a conventional pipe, and 35 mg vaporized DMT tends to be roughly equivalent to smoking 50 mg. This concept of “smoked equivalent” is important for creating the initial dosing parameters of the extended state experience.

### Example 6

### Intravenous Infusion to Achieve a Steady State—the Two Compartment Model

On the day of the psychedelic experience, time should be taken to have a follow-up discussion with the subject to ensure a productive mindset prior to the experience. Care should also be taken to provide an environment that is comfortable both physically and emotionally for the subject. A comfortable couch, or bedding in a softly lit room, with a quiet atmosphere, and meaningful decorations should be considered as elements that may be beneficial to a subject.

The “DMT space” can be overwhelming and difficult to navigate. Overshooting the levels in the CNS can be distressing and even traumatic. The subject's sense of time will usually be altered (dilated). Overshooting this state with intravenous infusion may be distressing until the DMT is either redistributed to another compartment, and or metabolized and/or eliminated.

1. The subject is welcomed; an initial verbal summary is taken. Concerns are voiced and the subject is introduced to the caregiver and any other observers. When the subject is ready, a site needs to be chosen for infusion. In the case of intravenous infusion, a vein should be prepped and accessed in a conventional manner. However, because of the concern for venous thrombophlebitis, intramuscular infusion should be considered the default route.

2. The subject will be asked to rest supine and will be connected to the infusion pump.

3. An initial bolus dose of 25 mg bolus for a 75 kg individual is given. This is the dose described in the original Strassman paper as well as the Gallimore and Strassman paper from 2016.

If the dose is too high, the experience may be overwhelming and/or distressing, but will rapidly abate due to redistribution of drug into the rest of the body over a steep concentration gradient.

Strassman et. al (2016) used DMT fumarate for single IV bolus infusion, and used 0.4 mg/kg as the “ideal” dose. This may be a good rule of thumb, but for a 75 kg male, this would be equivalent to 30 mg of DMT fumarate, which would be roughly equal to 22.5 mg of freebase DMT that was vaporized perfectly. In other words, an average size male of around 75 kg who routinely smokes 50 mg of DMT, or vaporizes 35 mg of DMT, may find that a bolus dose of 21 mg is not enough to be at an “ideal” experience level.

4. The computer-controlled pump initiates an infusion 2 minutes after the initial bolus, with the operator responsible for manually overriding the program to turn the pump off and on to maintain the subject at or near an “ideal” state, with the computer program recording and documenting each of these manual interventions. It should be appreciated that the pump can be turned off if at any time the subject starts to recognize that the experience may become challenging. Overshoot in this model is minimal, and within 3-5 minutes levels are declining. Documenting the user experience is critical for the model as point data for assumed “ideal and threshold” levels.

5. To determine the linear decrease, a second assumption, which is that when near steady state, the rate of infusion will be somewhere between 0.5 and 4.6 mg/min for a 25 mg smoked equivalent, and double this for a 50 mg smoked equivalent.

6. It will be assumed that the subject is at or near steady state at 20 minutes. This means there are 18 steps in one-minute intervals, starting with 4.2 mg/min and ending with 0.9 mg/min, in the 25 mg smoked equivalent case. So, the calculated delta for each step is the initial infusion rate Ri minus the final infusion rate Rf, divided by 18. In this example, the delta is approximately 0.10686.

7. Once steady state is achieved, there is no concentration gradient between the CNS and the peripheral tissues. The study can then be carried out for as long as the subject is comfortable and wishes to continue.

### Example 7

### Intramuscular Infusion to Achieve a Steady State—the Multi-Compartment Model

The same assumptions for IV infusion are made with IM infusion, a 75 kg individual who identifies smoking 25 mg of DMT as an ideal dose. A muscle should be chosen for access with the infusion needle. While any muscle will work, the most favorable option is probably the anterolateral thigh. This allows the patient to be positioned supine comfortably. If the anterolateral thigh is chosen, appropriate garments should be worn by the patient to expose the thigh in a manner that is suitable for access.

It will be appreciated by those of skill in the art that, in contrast to IV infusion, in the case of intramuscular infusion, the peripheral space is the first filled, before the CNS reaches the desired effect level.

With the patient lying supine, the anterolateral thigh should be prepped with either an alcohol swab, betadine, chlorhexidine or the like, to minimize the risk of infection. Once the skin has been suitably cleaned, intramuscular access with a needle should be obtained. If a straight needle is used, it can be difficult to secure to the skin. Therefore, a bend can be introduced to the hub of a needle. While a 90-degree bend would be ideal for allowing the tubing that attaches to the pump to be secured to the skin, bending a needle this much may decrease the patency of the lumen leading to occlusion or problems with the infusion pump not having enough driving pressure resulting in pump failure. Therefore a 30-60-degree bend is preferred, with the needle being placed within the quadriceps muscle slightly at an angle, allowing the hub of the needle to lay relatively flat with respect to the thigh, yet allowing good depth of penetration of the needle within the muscle. A 1.5 inch, 25 gauge needle seems ideal in this regard, but any gauge needle from 31 to 18 will work. A needle specifically manufactured for this purpose as described in the included claims would be ideal for this purpose. A 31 gauge needle may require excess pressure to be generated from the infusion pump, and an 18 gauge needle increases patient discomfort and the risk of bleeding unnecessarily.

1. The subject is provided with any information required, vitals are check and the subject relaxes supine.

2. The infusion pump is connected, and the catheter is inserted in the target muscle. Once the muscle has been accessed with the needle, the tubing should be attached, only after it has been primed (pre-filled) with DMT (or other drug) containing solution, so as not to inject excessive air into the muscle. Once the luer lock of the tubing has been attached to the needle hub, a sterile dressing can be placed over the needle and distal portion of the tubing to keep the infusion site clean and minimize the risk of infection.

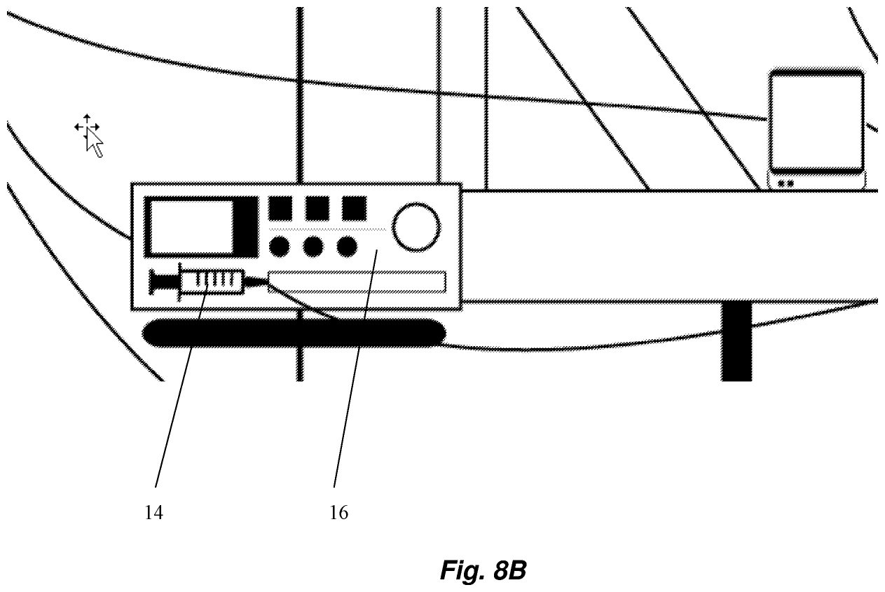

The proximal portion of the tubing should be attached to a syringe/catheter containing the DMT (or other drug) solution, and for this purpose, while any tubing would be suitable, microbore tubing is preferred to avoid wasted infusate. FIG. 8 illustrates a patient undergoing a psychotropic experience according to one embodiment of the invention. As illustrated the patient is lying supine connected to a computer which, in this illustration not only controls a diffusion pump but also is configured to acquire almost any data desirable. Such data can include temperature, blood oxygenation, brain function, blood pressure etc. FIG. 8, 10 computer, 12 computer connection to blood pressure compressor, 14 syringe, 16 syringe pump, computer connection to oximeter, 22 compressor connection to blood pressure cuff, 24 microbore tubing connecting cannula to syringe pump, 26 electroencephalogram leads, 28 blood pressure cuff. It will be appreciated by those of skill in the art that the data collection and control by the computer can include any number of other data sets as desired for example other monitors can include urinary catheter, pelvic thermistor and cardiac monitor to name a few.

3. A 25 mg initial bolus is given.

DMT fumarate is the salt form of N—N-dimethyltryptamine, allowing for water solubility. An aqueous solution of DMT fumarate can be prepared in a wide variety of concentrations, however around 25 mg/ml is preferred, as this allows for the addition of bicarbonate in quantities suitable to achieve an iso-osmolar preparation at near-physiologic pH.

There are many different types of programmable pumps which would be suitable for use in this application, however a syringe pump is preferred, allowing for precise control of drug delivery with the capacity to achieve high pressures required to overcome resistance from the length of microbore tubing and needle.

The programmable infusion pump (syringe pump) is controlled by a computer program. In the first pass administration, the rate constants are not known. Therefore in this example, only two inputs are required, the smoked equivalent dose that the patient prefers, and the maintenance dose.

In a 75 kg subject who prefers a smoked equivalent of 50 mg freebase N—N-dimethyltryptamine, the bolus dose would be approximately 1.7×50 mg or 85 mg. The maintenance dose in this case would be 0.09*50 mg or 4.5 mg/min. If a patient weighs significantly less than 75 kg, these doses (bolus and maintenance doses) should be scaled downward proportionately. If a patient weighs more than 75 kg, the dose should not be scaled up, as it is better to err on the side of too little drug than too much. Recognize that these doses are an educated guess, designed to create a pharmacologic dose curve that provides margin for error. Specifically, the bolus dose is chosen to be a little bit less than what is required to achieve an ideal peak effect by itself, and the maintenance dose is chosen to be a little higher than what is required to achieve steady state.

When the subject is ready, the bolus dose is administered over a short amount of time, preferably about 60 seconds, from time t=0 to t=1 minute. At t=2 minutes, the maintenance dose of 4.5 mg/min is started.

For this example, entire area under the curve from the IV example, which turns out to be 86.53 mg (25 mg initial bolus, 4.2 mg/min at t=2 min, and 2.28 mg at t=20 min). It is assumed that at time=2 min an infusion rate of 2.28 mg/min is started, which will continue from t=2 min to t=20 min. Beyond 20 minutes it is assumed that the subject is near steady state, but either way, the pump is turned on and off to maintain a near ideal level.

4. At time=2 min an infusion rate of 2.28 mg/min is started.

Of the 86.53 mg under the total curve, 2.28 mg*18 min (41.04 mg) will be administered via infusion after the initial bolus dose. This leaves 86.53 mg minus 41.04 mg as the initial bolus dose (45.49 mg). Again, a subject with a smoked equivalent preference of 50 mg would have numbers that are double these.

5. This infusion rate will continue from t=2 min to t=20 min (25 mg initial bolus, 4.2 mg/min at t=2 min, and 2.28 mg at t=20 min) is taken.

Within the first 2 minutes, the subject should notice the very first drug effects, such as a feeling of warmth, or perhaps a slight perceptual change. Typically, within minutes after the initial bolus dose, closed-eye visuals will begin. The DMT space will be described here as the experience of a full dose, where the subject feels as if he or she is in “another place.” Subjects may describe this as “a room” or “a scene.” Prior experience helps a subject understand what this is. A threshold dose will be described here as somewhere in between the very first noticeable effects, and something less than the full effect. At the threshold dose, the “DMT space” is becoming evident, but the visuals are only lightly perceptible. The threshold effect is typically experienced during the slow transition somewhere between 5 and 10 minutes, with the full effects typically noticed between 7 and 10 minutes depending on the dose and the patient's individual pharmacokinetics and rate constants. Note that it is believed that a threshold experience probably equates to a CNS concentration of roughly 60 ng/ml, and a full experience probably equates to a CNS concentration of roughly 100 ng/ml. What is important is the subjective experience rather than the exact CNS concentration. By recording the experience and timeline, the model can reproduce the same depth of experience by calculating the rate constants and continual or intermittent dosing required to achieve that same depth with no or little pump intervention.

Beyond 20 minutes it should be assumed that the subject is at or near steady state. At that time the pump can be turned on and off to maintain a near ideal level which can be maintained for the duration of the study.

Once the subject has achieved what they perceive to be the ideal level of depth of experience, the pump should be turned off, and the time recorded. After the pump has been shut off, there may be a slight intensification of effects that typically do not last for more than about 5 minutes. At the first notice of decreased effects, slightly below what the subject considers ideal, the pump is turned on, and the time is recorded. In this manner, the pump will be turned on and off as needed to maintain a relatively consistent state.

When either the observer or subject feels that the session should end, the pump is turned off. Because of the short half-life of DMT, the drug effects wear off rather quickly, typically fully abating within about an hour or less.

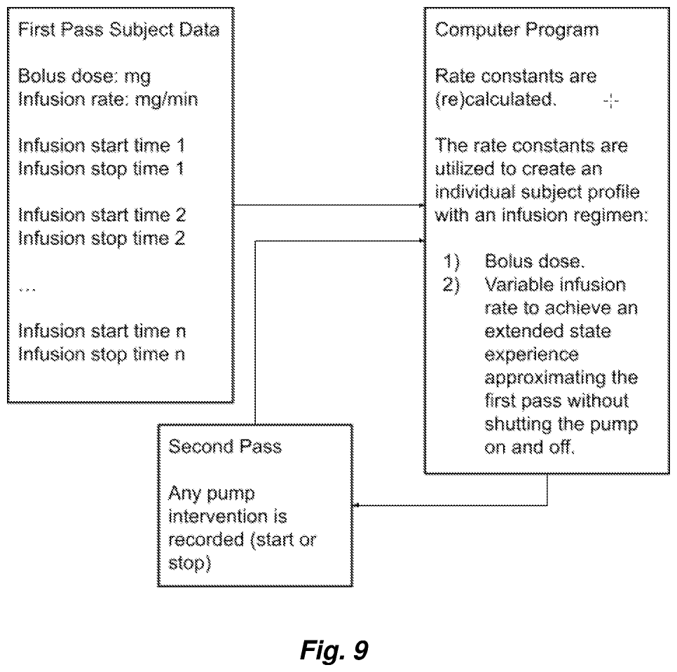

The recorded data of the time to threshold effects, time to ideal experience, and the subsequent on/off states of the pump are then input into the pump controller program, which then produces a profile for that subject. In any subsequent session, the subject will have a similar experience with minimal pump intervention. FIG. 9 provides a schematic of the computer-controlled data acquisition according to one embodiment of the invention.

In the case that the subject never reaches the ideal experience, then a subsequent first pass will be needed with the bolus and maintenance doses adjusted upward based on the observer's judgement, typically in increments of 10-25% or even up to 50% based on how short of the ideal experience the subject reported.

The needle and sterile dressing should be removed from the target, and a bandage should be applied to the site. The patient can change clothing, if required, at this time.

At the conclusion of the extended state psychedelic session, there should be a discussion with the subject about the experience (on the same day), to set the stage for follow-up interviews. Notes of the experience should be taken by the observer, for integration, which is the practice of working with the material that came up during the session for later non-drug sessions to help with a good therapeutic outcome in the case of therapy, or for deeper personal as well as scientific understanding in the case of research.

All publications and patents specifically mentioned herein are incorporated by reference for all purposes including describing and disclosing the chemicals, instruments, statistical analyses, and methodologies which are reported in the publications which might be used in connection with the invention. All references cited in this specification are to be taken as indicative of the level of skill in the art. Nothing herein is to be construed as an admission that the invention is not entitled to antedate such disclosure by virtue of prior invention.

The following paragraphs enumerated consecutively from 1 through 45 provide various additional aspects of the present invention. In one embodiment is a first paragraph:

1. A method for steady state infusion of a drug comprising:
calculating the rate constants for transport of a drug from an infusion site to an active site;
creating a computer deliverable program that calculates the rate constants suited to control an infusion pump;
providing an infusion pump controllable by the computer deliverable program; and
delivering the drug to a patient via the computer directed infusion pump at a desired flow rate or flow constant (k).

2. The method of paragraph 1, wherein the delivery is intravenous or intramuscular.

3. The method of paragraphs 1 or 2, where in the drug is a serotonergic drug.

4. The method of paragraphs 1 to 3, wherein the serotonergic drug is a psychedelic drug or a psychotropic drug.

5. The method of paragraphs 1 to 4, wherein intramuscular infusion comprises a needle having beveled tip, with a hole at the end, typical of needles used for intramuscular injection.

6. The method of paragraphs 1 to 5, wherein the needle comprises a number of side holes ranging from 1-100.

7. The method of paragraph 6, wherein the needle comprises 2-20 side holes.

8. The method of paragraphs 1 through 7, wherein the needle comprises curve at the base of the needle ranging from 0 to 90 degrees.

9. The method of paragraphs 1 through 8, wherein an initial bolus of the drug is delivered followed by the steady state delivery.

10. The method of paragraphs 1 through 9, wherein the computer deliverable program comprises a two-compartment model.

11. The method of paragraphs 1 through 9, wherein the computer deliverable program comprises a multicompartment model.

12. The method of paragraphs 1 through 11, wherein the pump is a miniature pump.

13. The method of paragraphs 1 through 12, wherein the pump is an implantable pump.

14. The method of paragraphs 1 through 13, wherein intramuscular means within the pectoralis, the gluteus, the calf, the rectus abdominus, the quadriceps or other muscle capable of maintaining an intramuscular needle.

15. A method of treating a mental health complaint according to any preceding paragraph comprising administering to a patient in need thereof:
a steady state concentration of a psychotropic compound,
wherein the steady state concentration is maintained by a by a computer controlled infusion pump;
wherein the infusion pump maintains the steady state concentration according to two compartment or multi-compartment kinetics.

16. The method of paragraphs 1 through 15, wherein the kinetics is first order.

17. The method of paragraphs 1 through 16, wherein the kinetics is second order.

18. The method of paragraphs 1 through 17, wherein the psychotropic compound is administered intravenously.

19. The method of paragraphs 1 through 18, wherein the psychotropic compound is administered intramuscularly.

20. The method of paragraphs 1 through 19, wherein a modified needle is used.

21. The method of paragraphs 1 through 20, wherein the modified needle is an indwelling cannula.

22. The method of paragraphs 1 through 21, wherein the needle comprises 2-20 side holes.

23. The method of paragraphs 1 through 22, wherein the mental health indication is: stress, depression, anxiety, insomnia, obsessive-compulsive disorder and addictive disorders.

24. The method of paragraphs 1 through 23, wherein the addictive disorders are: substance abuse.

25. The method of paragraphs 1 through 24, wherein the substance abuse is abuse of drugs, cigarettes, media, and alcohol.

26. An apparatus for administering a steady-state infusion of a drug comprising:
a computer assisted infusion device;
a computer program capable of driving the infusion device;
wherein the computer program calculates the rate constants of a patient using the infusion device;
wherein the infusion device delivers a steady-state infusion of drug to the patient.

27. The apparatus of paragraphs 1 through 26, wherein the infusion device delivers the drug intravenously or intramuscularly.

28. The apparatus of paragraphs 1 through 27, wherein intramuscular infusion comprises a modified needle.

29. The apparatus of paragraphs 1 through 28, wherein the modified needle comprises a beveled tip with a hole at the end.

30. The apparatus of paragraphs 1 through 29, wherein the needle comprises a number of side holes ranging from 2-100.

31. The apparatus of paragraphs 1 through 30, wherein the needle comprises curve at the base of the needle ranging from 0 to 90 degrees.

32. The apparatus of paragraphs 1 through 31, wherein the computer assisted infusion device infuses the drug with kinetics of a two-compartment model or a multi-compartment model.

33. A method for decreasing intravenous thrombophlebitis resulting from infusion of serotonergic compounds according to any of paragraphs 1 through 32 comprising the addition of heparin, or polyethylene glycol.

34. The method according to any of paragraphs 1 through 33, wherein the infusion is intravenous.

35. The method according to any of paragraphs 1 through 34, wherein the infusion is intramuscular.

36. A method for mitigating the effects of seratonergic drugs on thrombophlebitis according to any of paragraphs 1 through 35 comprising adding low concentration heparin in doses ranging from 0.1-100 IU per mL, comprising I.

37. The method according to any of paragraphs 1 through 36, wherein the heparin concentration is in the range of 1-10 IU per mL.

38. The method according to any of paragraphs 1 through 37, wherein the heparin is added to a maintenance line.

39. A method for decreasing venous injury and improving flow rate in a patient infused with a serotonergic compound according to any of paragraphs 1 through 38 comprising adding magnesium in doses ranging from 0.1 mg/mL to 100 mg/mL, to the infusate.

40. The method of paragraph 39, wherein the magnesium is added in the rage of about 1-2 mg/mL, added to the DMT infusion directly.

41. The method according to any of paragraphs 1 through 40, wherein the magnesium is added to a maintenance line.

42. A method for improving vein patency and dilution from blood flow as well as normal saline according to any of paragraphs 1 through 41, comprising preparing the vein to be cannulated with nitroglycerine ointment prior to gaining intravenous access.

43. A method for creating an injectable form of a serotonergic compound salt such as fumarate, or freebase of a serotonergic compound in a solution or suspension or emulsion according to any of paragraphs 1 through 42, comprising: an iso osmolar or near iso osmolar carrier, minimizing molecular interaction with the walls and endothelium of the peripheral vasculature, while maintaining water soluble or miscible properties as to not adversely affect the pharmacokinetics.

44. The method according to any of paragraphs 1 through 43, wherein the iso osmolar or near iso osmolar carrier comprises hyaluronic acid or polyethylene glycol.

45. A method for creating an injectable form of any other drug or substance that may cause venous irritation, to minimize the risk of thrombophlebitis, according to any of paragraphs 1 through 44 comprising the addition of nanoparticles or crystals in suspension, or similarly, chemotherapeutic drugs, anesthetic agents and the like.

While this invention has been described in conjunction with the various exemplary embodiments outlined above, various alternatives, modifications, variations, improvements, and/or substantial equivalents, whether known or that are or may be presently unforeseen, may become apparent to those having at least ordinary skill in the art. Accordingly, the exemplary embodiments according to this invention, as set forth above, are intended to be illustrative, not limiting. Various changes may be made without departing from the spirit and scope of the invention. Therefore, the invention is intended to embrace all known or later-developed alternatives, modifications, variations, improvements, and/or substantial equivalents of these exemplary embodiments. Those of skill in the art will recognize, or be able to ascertain, using no more than routine experimentation many equivalents to specific embodiment of the invention described specifically herein. Such equivalents are intended to be encompassed in the scope of the following claims.

What is claimed is:

1. A method for steady state infusion of a drug comprising:
calculating the rate constants for transport of a drug from an infusion site to an active site;
creating a computer deliverable program that calculates the rate constants suited to control an infusion pump;
providing an infusion pump controllable by the computer deliverable program; and
delivering the drug to a patient via the computer directed infusion pump at a desired flow rate or flow constant (k).

2. The method of claim 1, wherein the delivery, via a needle, is intravenous or intramuscular.

3. The method of claim 1, where in the drug is a serotonergic drug.

4. The method of claim 3, wherein the serotonergic drug is a psychedelic drug or a psychotropic drug.

5. (canceled)

6. The method of claim 2, wherein the needle comprises a number of side holes ranging from 1-100.

7. (canceled)

8. The method of claim 2, wherein the needle comprises curve at the base of the needle ranging from 0 to 90 degrees.

9. The method of claim 1, wherein an initial bolus of the drug is delivered providing kinetics for its steady state delivery.

10. The method of claim 1, wherein the computer deliverable program comprises a two-compartment model.

11. The method of claim 1, wherein the computer deliverable program comprises a multi-compartment model.

12.-13. (canceled)

14. The method of claim 2, wherein intramuscular means within the pectoralis, the gluteus, the calf, the rectus abdominus, the quadriceps or other muscle capable of accepting an intramuscular needle.

15. A method of treating a mental health complaint comprising administering to a patient in need thereof:
a steady state concentration of a psychotropic compound,
wherein the steady state concentration is maintained by a by a computer controlled infusion pump;
wherein the diffusion pump maintains the steady state concentration according to two compartment or multi-compartment kinetics.

16. The method of claim 15, wherein the kinetics is first order.

17. The method of claim 15, wherein the kinetics is second order.

18. The method of claim 15, wherein the psychotropic compound is administered intravenously or intramuscularly.

19.-25. (canceled)

26. An apparatus for administering a steady-state infusion of a drug comprising:
a computer assisted infusion device;
a computer program designed to drive the infusion device;
wherein the computer program calculates the rate constants of a patient using the infusion device;
wherein the infusion device delivers a steady-state infusion of drug to the patient.

27. The apparatus of claim 26, wherein the infusion device delivers the drug intravenously or intramuscularly.

28. The apparatus of claim 27, wherein intramuscular infusion comprises a modified needle.

29. (canceled)

30. The apparatus of claim 28, wherein the needle comprises a number of side holes ranging from 2-100.

31. The apparatus of claim 28, wherein the needle comprises curve at the base of the needle ranging from 0 to 90 degrees.

32. The apparatus of claim 26, wherein the computer assisted infusion device infuses the drug with kinetics of a two-compartment model or a multi-compartment model.

33.-45. (canceled)
